SEQUENCE LISTING (95 sequences)

SEQ ID NO: 1 - DNA, 6 bp, Artificial Sequence
  Synthetic sequence
gccacc

SEQ ID NO: 2 - DNA, 33 bp, Artificial Sequence
  Synthetic sequence
atgcatcaccatcatcaccaccatcaccaccat

SEQ ID NO: 3 - DNA, 600 bp, Artificial Sequence
  Synthetic sequence
gaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggc

SEQ ID NO: 4 - DNA, 600 bp, Artificial Sequence
  Synthetic sequence
tccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgag

SEQ ID NO: 5 - DNA, 3588 bp, Artificial Sequence
  Synthetic sequence
gccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccgccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggccc

SEQ ID NO: 6 - DNA, 1794 bp, Artificial Sequence
  Synthetic sequence
gccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggccc

SEQ ID NO: 7 - DNA, 1131 bp, Artificial Sequence
  Synthetic sequence
atgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagc

SEQ ID NO: 8 - DNA, 303 bp, Artificial Sequence
  Synthetic sequence
atgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggt

SEQ ID NO: 9 - DNA, 24 bp, Artificial Sequence
  Synthetic sequence
ctggaagttctgttccaggggccc

SEQ ID NO: 10 - DNA, 966 bp, Artificial Sequence
  Synthetic sequence
gccaccatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggccc

SEQ ID NO: 11 - DNA, 966 bp, Artificial Sequence
  Synthetic sequence
gccaccatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggccc

SEQ ID NO: 12 - DNA, 4361 bp, Artificial Sequence
  Synthetic sequence
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 13 - DNA, 4776 bp, Artificial Sequence
  Synthetic sequence
ggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgcc

SEQ ID NO: 14 - DNA, 5238 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 15 - DNA, 4839 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 16 - DNA, 6570 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 17 - DNA, 6570 bp, Artificial Sequence
  Synthetic Sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 18 - DNA, 5742 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 19 - DNA, 5742 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 20 - DNA, 5301 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 21 - DNA, 7032 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 22 - DNA, 7032 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcgccaccatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 23 - DNA, 135 bp, Artificial Sequence
  Synthetic sequence
ctggaagttctgttccaggggcccggatcccggtccgaagcgcgcggaattcaaaggcctacgtcgacgagctcactagtcgcggccgctttcgaatctagagcctgcagtctcgaggcatgcggtaccaagctt

SEQ ID NO: 24 - DNA, 64 bp, Artificial Sequence
  Synthetic sequence
gaagacttgatcacccgggatctcgagccatggtgctagcagctgatgcatagcatgcggtacc

SEQ ID NO: 25 - DNA, 129 bp, Artificial Sequence
  Synthetic sequence
ctggaagttctgttccaggggcccggatcccggtccgaagcgcgcggaattcaaaggcctacgtcgacgagctcactagtcgcggccgctttcgaatctagagcctgcagtctcgacaagcttgtcgag

SEQ ID NO: 26 - DNA, 76 bp, Artificial Sequence
  Synthetic sequence
ctgcagctcgctgcctcggagaacaacggaggtgaaggaattctgataatctagagcctgcagtctcgacaagctt

SEQ ID NO: 27 - DNA, 52 bp, Artificial Sequence
  Synthetic sequence
ggatccactctgcagctcgctgcctcggagaacaacggaggtgaaggaattc

SEQ ID NO: 28 - DNA, 27 bp, Artificial Sequence
  Synthetic sequence
tctagagcctgcagtctcgacaagctt

SEQ ID NO: 29 - DNA, 49 bp, Artificial Sequence
  Synthetic sequence
cactgagcgtcagaccccgtagaaaagatccgcgttgctggcgtttttc

SEQ ID NO: 30 - DNA, 58 bp, Artificial Sequence
  Synthetic sequence
ccagcaaaaggccaggaaccgtaaaaaggcaaaggatcttcttgagatcctttttttc

SEQ ID NO: 31 - DNA, 20 bp, Artificial Sequence
  Synthetic sequence
gcctttttacggttcctggc

SEQ ID NO: 32 - DNA, 22 bp, Artificial Sequence
  Synthetic sequence
gatcttttctacggggtctgac

SEQ ID NO: 33 - DNA, 62 bp, Artificial Sequence
  Synthetic sequence
catcaccaccatcaccaccatctggaagttctgttccaggggcccggatcccggtccgaagc

SEQ ID NO: 34 - DNA, 63 bp, Artificial Sequence
  Synthetic sequence
cttccagatggtggtgatggtggtgatgatggtgatgcatggtggcgcgcccgatggtgggac

SEQ ID NO: 35 - DNA, 70 bp, Artificial Sequence
  Synthetic sequence
tcataccgtcccaccatcgggcgcgccaccatgcatcaccatcatcaccaccatcaccaccatatgaaga

SEQ ID NO: 36 - DNA, 18 bp, Artificial Sequence
  Synthetic sequence
gctaccagctcctccagc

SEQ ID NO: 37 - DNA, 19 bp, Artificial Sequence
  Synthetic sequence
aactcctctagcgctggag

SEQ ID NO: 38 - DNA, 18 bp, Artificial Sequence
  Synthetic sequence
gggcccctggaacagaac

SEQ ID NO: 39 - DNA, 17 bp, Artificial Sequence
  Synthetic sequence
ggatcccggtccgaagc

SEQ ID NO: 40 - DNA, 17 bp, Artificial Sequence
  Synthetic sequence
gcgcccgatggtgggac

SEQ ID NO: 41 - DNA, 21 bp, Artificial Sequence
  Synthetic sequence
catatggtggtgatggtggtg

SEQ ID NO: 42 - DNA, 50 bp, Artificial Sequence
  Synthetic sequence
gccaccatgcatcaccatcatcaccaccatcaccaccatatgggaagcct

SEQ ID NO: 43 - DNA, 51 bp, Artificial Sequence
  Synthetic sequence
gccgctgctgctgtcgtcgtcctcctcctcacctccgatttgttccctgtg

SEQ ID NO: 44 - DNA, 51 bp, Artificial Sequence
  Synthetic sequence
gctgtcttcgctgtcctcgctgtcttcggaacctccgatttgttccctgtg

SEQ ID NO: 45 - DNA, 20 bp, Artificial Sequence
  Synthetic sequence
tccgaagacagcgaggacag

SEQ ID NO: 46 - DNA, 54 bp, Artificial Sequence
  Synthetic sequence
ctggaagttctgttccaggggcccggatccatgaataacggttctggtcgatac

SEQ ID NO: 47 - DNA, 56 bp, Artificial Sequence
  Synthetic sequence
tatgatcctctagtacttctcgacaagctttcaatgtggatttttcctctcaaacc

SEQ ID NO: 48 - DNA, 52 bp, Artificial Sequence
  Synthetic sequence
ctggaagttctgttccaggggcccggatccatggatttatctgctcttcgcg

SEQ ID NO: 49 - DNA, 48 bp, Artificial Sequence
  Synthetic sequence
tatgatcctctagtacttctcgacaagctttcagtagtggctgtgggg

SEQ ID NO: 50 - DNA, 52 bp, Artificial Sequence
  Synthetic sequence
ctggaagttctgttccaggggcccggatccatgccaaggaggacaaaaaagg

SEQ ID NO: 51 - DNA, 54 bp, Artificial Sequence
  Synthetic sequence
tatgatcctctagtacttctcgacaagcttctaagagtcctgctcaatcatatc

SEQ ID NO: 52 - DNA, 54 bp, Artificial Sequence
  Synthetic sequence
ctggaagttctgttccaggggcccggatccatggaccaaagagaaattctgcag

SEQ ID NO: 53 - DNA, 54 bp, Artificial Sequence
  Synthetic sequence
tatgatcctctagtacttctcgacaagcttttaaatattccaagttggtggtgg

SEQ ID NO: 54 - DNA, 57 bp, Artificial Sequence
  Synthetic sequence
ctggaagttctgttccaggggcccggatcccaacaaacgttgtcttcgttctttatg

SEQ ID NO: 55 - DNA, 58 bp, Artificial Sequence
  Synthetic sequence
tatgatcctctagtacttctcgacaagctttcatttcaaagtttctaaacgtttatag

SEQ ID NO: 56 - DNA, 55 bp, Artificial Sequence
  Synthetic sequence
tcataccgtcccaccatcgggcgcgccaccatgcatcaccatcatcaccaccatc

SEQ ID NO: 57 - DNA, 20 bp, Artificial Sequence
  Synthetic sequence
ctggaagttctgttccaggg

SEQ ID NO: 58 - DNA, 48 bp, Artificial Sequence
  Synthetic sequence
cacttgcagttcctcgcagtcgtcttccatggatccgggcccctggaa

SEQ ID NO: 59 - DNA, 48 bp, Artificial Sequence
  Synthetic sequence
gtccttcacgtcgatgtccatgtcgggcatggatccgggcccctggaa

SEQ ID NO: 60 - protein, 9 aa, Artificial Sequence
  Synthetic sequence
EEEDDDSSS

SEQ ID NO: 61 - protein, 9 aa, Artificial Sequence
  Synthetic sequence
DDDEEESSS

SEQ ID NO: 62 - protein, 9 aa, Artificial Sequence
  Synthetic sequence
SSSEEEDDD

SEQ ID NO: 63 - protein, 100 aa, Artificial Sequence
  Synthetic sequence
EEEDDDSSSGGEEESSSDDDGGDDDEEESSSGGDDDSSSEEEGGSSSEEEDDDGGSSSDDDEEEGGEEEDDDSSSGGDDDEEESSSGGSSSDDDEEEGGG

SEQ ID NO: 64 - protein, 200 aa, Artificial Sequence
  Synthetic sequence
EEEDDDSSSGGEEESSSDDDGGDDDEEESSSGGDDDSSSEEEGGSSSEEEDDDGGSSSDDDEEEGGEEEDDDSSSGGDDDEEESSSGGSSSDDDEEEGGGEEEDDDSSSGGEEESSSDDDGGDDDEEESSSGGDDDSSSEEEGGSSSEEEDDDGGSSSDDDEEEGGEEEDDDSSSGGDDDEEESSSGGSSSDDDEEEGGG

SEQ ID NO: 65 - protein, 204 aa, Artificial Sequence
  Synthetic sequence
EEEDDDSSSGGEEESSSDDDGGDDDEEESSSGGDDDSSSEEEGGSSSEEEDDDGGSSSDDDGEEEGGEEEDDDSSSGGDDDEEESSSGGSSSDDGDEEEGGGEEEDDDSSSGGEEESSSDDDGGDDDEEESSSGGDDDSSSEEEGGSSSEEEDDDGGSSSDDDEEGEGGEEEDDDSSSGGDDDEEESSSGGSSSDDGDEEEGGG

SEQ ID NO: 66 - protein, 200 aa, Artificial Sequence
  Synthetic sequence
EEEDDDSSSGGESSSDDDGGDDDEESSSGGDDDSSSEEEGGSSSEEDDDGGSSSDDDGEEEGGEEEDDDSSSGGDDDESESSSGGSSSDEEGGGESEGEDDDSSSGGEGDSSSDDDGGDDDEEGESSSGGDDDSSSEEEGGSSSEEEDDDGGSSSDDDEGEEGGEEEDDDSSSGGDDDEEGESSSGGSSSDDGDEEEGGG

SEQ ID NO: 67 - DNA, 1788 bp, Artificial Sequence
  Synthetic sequence
atgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggccc

SEQ ID NO: 68 - DNA, 1788 bp, Artificial Sequence
  Synthetic sequence
atgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggccc

SEQ ID NO: 69 - DNA, 960 bp, Artificial Sequence
  Synthetic sequence
atgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggccc

SEQ ID NO: 70 - DNA, 960 bp, Artificial Sequence
  Synthetic sequence
atgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggccc

SEQ ID NO: 71 - DNA, 927 bp, Artificial Sequence
  Synthetic sequence
atgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggccc

SEQ ID NO: 72 - DNA, 927 bp, Artificial Sequence
  Synthetic sequence
atgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggccc

SEQ ID NO: 73 - DNA, 927 bp, Artificial Sequence; identical to SEQ ID NO: 71
  Synthetic sequence

SEQ ID NO: 74 - DNA, 927 bp, Artificial Sequence; identical to SEQ ID NO: 72
  Synthetic sequence

SEQ ID NO: 75 - DNA, 4833 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 76 - DNA, 6564 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 77 - DNA, 6564 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 78 - DNA, 5736 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 79 - DNA, 5736 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 80 - DNA, 6531 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 81 - DNA, 6531 bp, Artificial Sequence
  Synthetic sequence
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 82 - DNA, 5295 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 83 - DNA, 7026 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 84 - DNA, 7026 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 85 - DNA, 6198 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggtgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 86 - DNA, 6198 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgcatcaccatcatcaccaccatcaccaccatatgggaagcctccaggatagcgaagtcaaccaagaagccaagccagaagtgaagccagaagtgaagccagaaacacacatcaacctcaaggtgagcgatggttcctccgagatcttcttcaagatcaagaagaccactcccctgcgtcgcctcatggaggctttcgccaagcgtcagggcaaggaaatggactccttgacattcctgtacgatggcatcgaaatccaggctgaccaaactcctgaggacttggacatggaggacaacgacatcatcgaggctcacagggaacaaatcggaggttccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 87 - DNA, 6993 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagcgaggaggaggacgacgacagcagcagcggcggcgagtcatctagcgacgacgacggcggagacgacgacgaagaatccagcagcggaggtgacgatgactcctctagcgaggaagagggtggctcatcgtccgaagaggatgacgatggaggttctagctcagacgatgacggcgaagaggaaggcggagaggaagaggatgacgattcgtcctctggtggcgacgatgacgaatccgagagctcatcgggaggttcctctagcgacgaagagggcggtggtgaatccgagggagaggatgacgattcatcgtccggcggagagggtgactcctcctcagacgatgacggtggcgatgacgatgaagagggcgagtcgtcctctggaggtgacgatgacagctcatcggaagaggaaggcggttcctcctccgaagaggaggatgacgatggtggctcatcgtcagacgatgacgagggcgaagagggaggtgaagaggaagatgacgactcctcttctggtggagacgacgacgaggaaggcgagtcatctagcggtggctcctcttccgacgacggagacgaggaagagggaggtggcctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 88 - DNA, 6993 bp, Artificial Sequence
  Synthetic sequence
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcatgaagactgaagagggcaagctcgttatctggatcaacggcgacaagggctacaacggactcgctgaagtgggcaagaagttcgagaaggacactggcatcaaggtgacagtcgagcaccccgataagttggaggaaaagttccctcaggtcgctgctaccggcgacggacctgatatcatcttctgggctcacgacaggttcggtggatacgctcagtccggactgctcgctgagatcacacctgacaaggccttccaagataagctctacccattcacctgggacgctgtgagatacaacggcaagctgatcgcctaccccatcgccgtcgaggctttgtcactgatctacaacaaggacttgctgcccaacccccctaagacatgggaggaaatccctgctctcgataaggaattgaaggctaagggcaagtccgccctgatgttcaacctccaggagccttacttcacttggccactgatcgctgccgacggaggttacgccttcaagtacgagaacggcaagtacgacatcaaggatgttggcgtggacaacgctggtgccaaggctggcctcactttcttggtggatctgatcaagaacaagcacatgaacgctgacacagattactctatcgccgaagctgccttcaacaagggagagaccgctatgactatcaacggtccatgggcctggtctaacatcgacaccagcaaggtcaactacggcgtcacagttctgcccaccttcaagggacagccttccaagccattcgtgggcgtcctctccgctggaatcaacgctgcctctcctaacaaggagctcgccaaggaattcttggagaactacctcttgactgacgaaggtttggaggctgtcaacaaggataagcccctgggcgccgttgctctcaagtcctacgaggaagagctggctaaggaccctcgcatcgctgccaccatggaaaacgcccagaagggagagatcatgccgaacatcccccaaatgtctgccttctggtacgctgttcgtactgccgtgatcaacgctgctagcggtagacagaccgtggacgaggctctgaaggatgcccaaactaactcctctagcgctggaggagctggtagctccgaagacagcgaggacagcgaagacagcgaggacagcgaagacagcgaggactccgaagattcagaggactccgaggattccgaagactccgaggattctgaagacagcgaggattcagaagactcggaggattccgaagactctgaggatagcgaagactcagaggattcggaagattctgaagactccgaggattccgaggactccgaggattctgaggactctgaggactccgaagactccgaggattcagaggattcggaagactctgaagactccgaggacagcgaagactccgaggactctgaagactctgaagattccgaagactccgaagactcggaagattcggaagattctgaggactcagaggattccgaagactcggaggattctgaagactctgaggattccgaagacagcgaagattccgaggattcggaagattcagaagactctgaagacagcgaggactcagaggactctgaggactcagaggacagcgaggactcagaagattctgaagattccgaggatagcgaggattcggaggactccgaagattcggaagattcggaggactcagaagactccgagctggaagttctgttccaggggcccggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 89 - DNA, 51 bp, Artificial Sequence
  Synthetic sequence
gattattcataccgtcccaccatcgggcgcatgaagactgaagagggcaag

SEQ ID NO: 90 - DNA, 17 bp, Artificial Sequence
  Synthetic sequence
gcgcccgatggtgggac

SEQ ID NO: 91 - DNA, 64 bp, Artificial Sequence
  Synthetic sequence
tcataccgtcccaccatcgggcgcatgcatcaccatcatcaccaccatcaccaccatatgaaga

SEQ ID NO: 92 - DNA, 49 bp, Artificial Sequence
  Synthetic sequence
tcataccgtcccaccatcgggcgcatgcatcaccatcatcaccaccatc

SEQ ID NO: 93 - protein, 200 aa, Artificial Sequence
  Synthetic sequence
SEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSE

SEQ ID NO: 94 - protein, 200 aa, Artificial Sequence
  Synthetic sequence
EDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSED

SEQ ID NO: 95 - protein, 200 aa, Artificial Sequence
  Synthetic sequence
DSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDSEDS

(19) United States
(12) Patent Application Publication
(10) Pub. No.: US 2021/0139920 A1
(43) Pub. Date: May 13, 2021

(54) SOLUBILITY ENHANCING PROTEIN EXPRESSION SYSTEMS

(71) Applicant: Indiana University Research and Technology Corporation, Indianapolis, IN (US)

(72) Inventors: Yuichiro Takagi, Indianapolis, IN (US); Tsuyoshi Imasaki, Indianapolis, IN (US)

(73) Assignee: Indiana University Research and Technology Corporation, Indianapolis, IN (US)

(21) Appl. No.: 16/641,175

(22) PCT Filed: Aug. 21, 2018
(86) PCT No.: PCT/US2018/047193

§ 371 Date: Feb. 21, 2020

Related U.S. Application Data
(60) Provisional application No. 62/548,247, filed on Aug. 21, 2017.

(51) Int. Cl. C12N 15/70; C07K 7/06; C12N 15/62

(52) U.S. Cl. CPC C12N 15/70; C07K 7/06; C12N 15/625; C07K 2319/50; C07K 2319/22; C07K 2319/24; C07K 2319/21

(57) ABSTRACT

Embodiments disclosed herein provide compositions, methods, and uses for solubility-enhancing protein (SEP) tags. Certain embodiments provide expression vectors for the production of a soluble protein or polypeptide of interest (i.e., a target protein) having a molecular mass of about 100 kDa or greater. In some embodiments, the SEP tags enable expression of large and often difficult to express proteins, with yields appropriate for further protein study. Also described are nucleic acid cassettes that include a SEP tag, and fusion proteins expressed from the SEP expression vectors or nucleic acid cassettes. Kits including SEP expression vectors are also provided.

### CROSS-REFERENCE TO RELATED APPLICATIONS

This PCT application claims priority to U.S. Provisional Patent Application No. 62/548,247, filed Aug. 21, 2017, the disclosure of which is incorporated herein by reference in its entirety.

### STATEMENT OF GOVERNMENTAL SUPPORT

This invention was made with government support under GM111695 awarded by National Institutes of Health and MCB1157688 awarded by National Science Foundation. The government has certain rights in the invention.

### FIELD

Various aspects and embodiments disclosed herein relate generally to compositions, methods, and uses for generating, expressing, and synthesizing a soluble form of a protein using a solubility-enhancing protein assisted protein expression (“SEP”) system. Solubility-enhancing protein assisted protein expression systems for use in E. coli expression systems (“eSEP” systems) are disclosed.

### SEQUENCE LISTING

The instant application contains a Sequence Listing which has been submitted via EFS-Web and is hereby incorporated by reference in its entirety. The ASCII copy, created on Aug. 15, 2018, is named IURTC-2016-003_5 T25, and is 249,935 bytes in size.

### BACKGROUND

The ability to generate high levels of recombinant protein expression is crucial in both the biopharmaceutical industry as well as in basic research. Generally, large amounts of specific proteins are required for such purposes, including for biochemical characterization of the protein, structural studies, drug discovery and development, gene therapy, subunit vaccine production, and reagent use.

Development of recombinant protein expression technologies has been one of the cornerstones in modern molecular biology. Several recombinant expression systems have been developed to produce recombinant proteins. Expression systems based on expression in mammalian, bacterial, yeast, plant, and insect cells are widely used for producing recombinant protein. While each expression system has its advantages, one common problem is that it is difficult to efficiently express large proteins having molecular weights of greater than about 100 kDa, in a soluble form with acceptable yields. In some cases, recombinant expression often leads to precipitation of the target protein as an insoluble mass in inclusion bodies in the host cell.

Large proteins often play essential roles in human biology. Their mutations are frequently associated with human diseases. Therefore, solving this fundamental bioengineering problem is critical for future biomedical and pharmaceutical studies and therapeutics.

### SUMMARY

Embodiments disclosed herein provide compositions, methods, and uses for solubility-enhancing protein (SEP) tags. Certain embodiments provide expression vectors for the production of a soluble protein or polypeptide of interest (target protein) having a molecular mass of about 100 kDa or greater. In some embodiments, the target protein has a molecular mass of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater. In some embodiments, the SEP tags enable expression of large and often difficult to express proteins, with yields appropriate for further protein study. Also described are nucleic acid cassettes that include an SEP tag, and fusion proteins expressed from the SEP expression vectors or nucleic acid cassettes. Kits including SEP expression vectors are also provided. In some embodiments, one or more solubility-enhancing polypeptide tags described herein can be encoded by a single expression vector.

In certain aspects, an expression vector including a vector backbone and at least one polynucleotide encoding a solubility-enhancing polypeptide, where the solubility-enhancing polypeptides is an AP tag, an SED tag, or a combination thereof. AP and SED tags are engineered polypeptides capable of increasing the production of soluble target proteins. In addition to the at least one polynucleotide encoding a solubility-enhancing polypeptide, expression vectors can further include one or more additional polynucleotide sequences, such as a multiple cloning site; a protein tag such as an affinity protein tag, a solubility enhancing protein tag other than AP or SED, and yield-improving protein tags; one or more promoters; and a protease recognition sequence. In some embodiments, the expression vector can be based on a mammalian vector backbone, a bacterial vector backbone, or a viral (e.g., baculovirus) vector backbone.

Other aspects described herein provide methods for expressing and producing a soluble target protein. The methods include providing an expression vector described herein and expressing the target protein from the expression vector. Expression of the vector can occur in an appropriate expression system, such as those derived from bacteria, yeast, baculovirus/insect, mammalian, or plant cells. In some embodiments, the methods can be used to produce large (100 kDa or greater) target proteins in a soluble form. The methods can further include isolating and purifying the expressed target protein. In certain embodiments, the target protein will be expressed as a recombinant protein, with the SEP or AD tag attached. Where the recombinant protein includes a protease recognition sequence between the solubility enhancing protein and the target protein, the recombinant protein can be cleaved to separate the target protein from the solubility enhancing protein.

Yet other aspects provide kits that include an expression vector encoding an AP or an SED tag and a cloning site suitable for cloning a polynucleotide encoding a target protein. Using a kit described herein, a user can clone into the vector at the cloning site a polynucleotide encoding a selected target protein. In some embodiments, the target protein is a large polypeptide (100 kDa or greater). The kits can allow for the efficient production of such large target proteins in a soluble form.

### BRIEF DESCRIPTION OF THE DRAWINGS

The following drawings form part of the instant specification and are included to further demonstrate certain aspects of particular embodiments herein. The embodiments may be better understood by reference to one or more of these drawings in combination with the detailed description presented herein.

FIG. 1A is a schematic diagram of SEP tags, according to an embodiment of the present disclosure. SEP0 tag comprises 10× histidine tag, 3C protease site, and an open reading frame of a target gene. SEP1 and SEP2 tags comprise 10× histidine tag, maltose-binding protein (MBP), solubility-enhancing protein (SEP1=AP; SEP2=SED) followed by a 3C protease site and an open reading frame of a target gene.

FIG. 1B is a diagram representing overall SEP tag function, according to an embodiment of the present disclosure. Large problematic target proteins tend to become insoluble when recombinantly expressed, as shown on the left. When fused with a SEP tag, these proteins can be recombinantly expressed in soluble forms, as shown on the right.

FIG. 1C is a diagram representing recombinant target protein purification scheme using the SEP system, according to an embodiment of the present disclosure. SEP tag fusion proteins can be captured by affinity column chromatography, and target proteins eluted by on-column digestion with 3C protease.

FIGS. 2A-2B are diagrams of representative SEP vectors, according to an embodiment of the present disclosure. Each of the representative vectors comprise replication origins (pMB1, f1 Ori), antibiotic resistant (Amp R, Gen R), promoters (polh, p10), terminators, affinity tags (10 His, MBP), a solubility-enhancing domain (AP or SED), Tn5 transposition sequences (Tn7R and Tn7L), 3C protease cleavage site (3C protease site), and multiple cloning sites (MCS (SEQ ID NO: 23), MCS1 (SEQ ID NO: 24), and MCS2 (SEQ ID NO: 25)). SEP0 is the base vector bearing only a 10×Histidine tag. SEP1 further includes an MBP-AP tag, while SEP2 includes an MBP-SED tag, each designed to improve solubilization and affinity purification of a target protein. SEP single vectors contain a single MCS for expression of a single gene. SEP dual vector contains two MCSs for simultaneous expression of two genes. MCS sequences and their unique restriction enzyme sites are shown.

FIGS. 3A-3C are photographs representing recovery of soluble target protein using the SEP system, according to an embodiment of the present disclosure. Eight different tags, 10×His, SUMO, GST, MBP, AP, SED, MBP-AP, and MBP-SED were fused to the N-terminus of NRDP1 or NRPD2, and the each fusion protein was expressed in a 50 ml Hi5 cell culture by infecting cells with a corresponding recombinant baculovirus. At the end of expression, cells were harvested, lysed, insoluble fractions (P: pellet) and soluble fraction (S: sup) were separated by centrifugation followed by immunobloting using anti-NRPD1 antibody (3A, lanes 1-12, 3C lanes 25-30), or anti-NRPD2 antibody (3B, lanes 13-24, 3C, lanes 31-36). FIG. 3A) The effect of 10×His, SUMO, GST, MBP, AP, SED tags for solubility of NRPD1 (lanes 1-12). FIG. 3B) The effect of 10×His, SUMO, GST, MBP, AP, SED tags for solubility of NRPD2 (lanes 13-24). FIG. 3C) The effect of MBP, MBP-AP, MBP-SED fusion tags for solubility of NRPD1 (lanes 24-30) and NRPD2 (lanes 31-36).

FIGS. 4A-4B are sequences of SEP tags and their predicted secondary structure, according to an embodiment of the present disclosure. FIG. 4A) Amino acid sequence of Acidic Patch tag (AP; SEQ ID NO: 66) on top, with the predicted secondary structure of each amino acid residue displayed below the sequence. FIG. 4B) Amino acid sequence of SED tag (SEQ ID NO: 93) on top, with the predicted secondary structure of each amino acid residue displayed below the sequence.

FIG. 5 is a photograph of an SDS-PAGE gel of purified His tagged (control) or SEP tagged (AP or SED) proteins, according to an embodiment of the present disclosure: yeast Med12, human LRRK2, DNA-PK, BRCA1, mTor, human lymphoid-specific protein tyrosine phosphatase (Lyp), and Drosophila CTCF protein. His tagged (control) or SEP-tagged 8 proteins described above were individually expressed in Hi5 cells using the baculovirus harboring the gene encoding 10×His, MBP-AP (or SED)-tagged protein, and affinity purified using either Ni column for His tagged proteins, and amylose column for SEP tagged proteins. The fractions from Ni or amylose column were analyzed by SDS-PAGE and expression levels of each tag were compared side by side for each protein. Arrow indicates SEP-fusion proteins.

FIGS. 6A-6B are photographs of culture plates representing increased pSEPa vector integration efficiency relative to pSEPb vectors, according to an embodiment of the present disclosure. For all pSEPb vectors, the pUC1 origin of pFastBac1 (Invitrogen) was replaced with pMB1 origin from pRS322 (Addgene). However, the pSEPb vectors displayed low integration efficiency. To improve integration efficiency, pSEP1 (FIG. 6A) and pSEP2 (FIG. 6B) were remade using the original origin of replication in pFastBac1, resulting in the pSEPa vectors. Utilizing pFastBac1's original origin of replication resulted in a marked improvement in integration efficiency, as visualized by the increased number of white colonies (indicating integration) over blue colonies (no integration).

FIG. 7A represents a schematic diagram of SEP tags, according to an embodiment of the present disclosure (e.g., SEP20, SEP21, SEP22, and SEP23 tags). Each tag comprises maltose-binding protein (MBP), 3C protease site and solubility-enhancing protein (AP or SED) followed by an open reading frame of a target gene.

FIG. 7B illustrates SEP tag functions. Large and problematic proteins (e.g., >100 kDa) tend to become insoluble when expressed, as depicted on the left. When fused with an SEP tag, these large proteins can be generated in soluble forms, as depicted on the right.

FIG. 7C illustrates a representative purification scheme using SEP tags. The SEP tag fusion proteins can be captured by amylose affinity column via the MBP moiety. The AP or SED fusion protein can be eluted by on-column digestion with 3C protease, resulting in removal of the MBP moiety.

FIGS. 8A-8D are exemplary vector maps of SEP tag vectors (pSEP20-pSEP23). The SEP vectors were designed to express large proteins or protein complexes that are difficult to produce. This is achieved by adding solubility-enhancing-protein, AP or SED, to the N-terminus of the target protein. Each exemplary SEP vector includes replication origins (pMB1, f1 ori), antibiotics resistance (Amp R, GenR), promoters (polh), terminators, affinity tags (MBP), a solubility-enhancing domain (AP or SED), Tn5 transposition sequences (Tn7R and Tn7L), 3C protease cleavage site (3C protease site), and multi cloning sites (MCSs). pSEP20 (FIG. 8A) contains MBP-3C-AP tag, and pSEP21 (FIG. 8B) contains MBP-3C-SED tag. 3C protease site was placed in between MBP and AP or SED such that MBP can be removed by 3C protease digestion, resulting in yielding AP or SED fusion protein. pSEP22 (FIG. 8C) contains MBP-3C-AP tag, as well as TEV site and Twin-Strep tag as the C-terminus tag, and pSEP23 (FIG. 8D) contains MBP-3C-SED tag, as well as TEV site and Twin-Strep tag as the C-terminus tag. Maps were generated by ApE.

FIG. 9A is a photograph of an SDS-PAGE gel of purified AP-RPS5 fusion protein. The open reading frame of the RPS5 gene was sub-cloned into BamHI and Hind III sites of pSEP20 vector followed by generation of a recombinant baculovirus. MBP-3C-AP-RPS5 fusion protein was expressed in Hi5 insect cells. The fusion protein was captured by an amylose column and AP-RPS5 was eluted by digestion with 3C protease. AP-RPS5 fusion protein is indicated by the arrow. M: molecular weight marker: size of each band is indicated (kDa) on the left.

FIG. 9B is a photograph of a negative stain electron micrograph depicting an AP-RPS5 protein preparation. Note the uniform-sized circular particles of approximately 10 nm in diameter.

FIG. 10 is a schematic diagram of SEP tags that can be used in E. coli expression systems, according to an embodiment of the present disclosure (e.g., SEP5e and SEP6e). Each tag comprises maltose-binding protein (MBP), solubility-enhancing protein (AP or SED) followed by 3C protease site and open reading frame of a target gene.

FIG. 11A-11H are exemplary vector maps of SEP tag vectors that can be used in E. coli expression systems (“eSEP” vectors). eSEP vectors are designed for expression of large and problematic proteins in E. coli. Each of the eSEP vectors comprises replication origin (pBR322 or p15A), antibiotics resistance (Amp R, Clm R, or Spec R), tac promoter, terminators, affinity tag (MBP), an eSEP solubilization domain (APe, SEDe), 3C protease cleavage site (3C protease site), and multi cloning sites (MCS). pSEP5e has and MBP-AP tag, and pSEP6e has and MBP-SED tag to facilitate target protein solubilization and affinity purification. Maps were generated by ApE.

FIG. 12 presents two photographs of SDS-PAGE gels of purified SEP-tagged plant NRPD1 (left) and NRPD2 (right) subunits. SEP-tagged plant NRPD1 or NRPD2 was individually expressed in bacteria in SEP fusion protein forms: MBP-AP (or SED)-NRPD1 or MBP-AP (or SED)-NRPD2, and affinity purified using amylose resin. The fractions from amylose column were analyzed by SDS-PAGE: MBP-AP-NRPD1 (lane 2); MBP-SED-NRPD1 (lane 3) on the left; MBP-AP-NRPD2 (lane 5); MBP-SED-NRPD2 (lane 6) on the right. M: molecular weight marker (lanes 1, 4), size of each band is indicated (kDa) on the left. (*): MBP-AP or MBP-SED tag alone; (**): MBP alone

### DETAILED DESCRIPTION

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step).

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

### Solubility Enhancing Peptides

Certain embodiments provide solubility-enhancing polypeptides (SEPs). The SEPs can be used to express large recombinant proteins, e.g., those proteins having a molecular weight of about 100 kDa or greater. In some embodiments, target proteins have a molecular weight of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater. In some embodiments, the SEPs can be used to express large recombinant proteins in existing expression systems. In some embodiments, the SEPs can be used to express large recombinant proteins in bacterial (e.g., E. coli) expression systems.

In some embodiments, the SEPs can be used to express large recombinant proteins in a soluble form. Protein solubility is important to all scientists who work with protein in solution, including structural biologists and those in the pharmaceutical industry, and is a common problem with recombinant protein expression. Structural studies and pharmaceutical applications such as drug discovery and development, and protein therapeutic development often require high-concentration protein samples. With insoluble expressed proteins being predominantly incorporated into inclusion bodies, it can take significant effort—if possible at all—to get protein from the inclusion bodies to the soluble fractions, making high-concentration protein samples difficult to produce.

Some embodiments provide expression vectors that encode a solubility-enhancing polypeptide described herein and at protein of interest. The expression vectors can be used to express and produce large recombinant proteins, where the solubility-enhancing polypeptide is linked to a protein of interest (FIG. 1A). In certain embodiments, the produced recombinant protein has increased solubility and stability relative to a target protein expressed and produced without the benefit of the solubility-enhancing polypeptide.

Large recombinant proteins having molecular weights of about 100 kDa or greater are difficult to produce. By examining the expression of the large proteins DNA-directed RNA polymerase IV subunit (NRPD1) and DNA-directed RNA polymerase IV subunit NRPD2, it was found that the issue in isolating significant amounts of recombinant proteins stemmed from the low solubility of the expressed proteins. These two proteins are the two largest subunits of plant RNA polymerase IC (Pol IV), which plays a critical role in gene silencing in plants. Both NRPD1 and NRPD2 are recognized as being very difficult to express

In accordance with some embodiments, 10×His-tagged NRPD1 and NRPD2 were individually expressed in insect cells using a baculovirus and/or bacterial (e.g., E. coli) expression vector system. Expression levels were determined by immunoblotting using anti-NRDP1 and NRDP2 antibodies. While both tagged proteins were expressed in a relatively large quantity (FIG. 3A, lane 1; FIG. 3B, lane 13), all recovered protein was insoluble. No soluble NRDP1 or NRDP2 was detected (FIG. 3A, lane 2; FIG. 3B, lane 14).

Affinity tags including small ubiquitin-related modifier (SUMO), glutathione S-transferase (GST), and maltose-binding protein (MBP) increase the solubility of the protein to which they are fused when expressed in bacterial expression systems. To test the ability of these tags to improve expression of soluble protein a baculovirus expression system, NRPD1 and NRDP2 were tagged with SUMO, GST, or MBP, and expressed in insect cells. None of the affinity tags tested improved solubility of either NRDP1 or NRDP2. All protein was insoluble (FIG. 3A, lanes 3, 5, and 7; FIG. 3B, lanes 15, 17, and 19).

Two polypeptides were engineered to improve the solubility of large target proteins. The two engineered polypeptides were generated and tested: a tag termed “Acid Patch” (AP), and a tag termed “SED.” Both novel tags comprise acidic amino acids glutamic acid (E), aspartic acid (D), and serine (S).

Certain embodiments provide an “Acid Patch” (AP) solubility tag. In some embodiments, the AP tag can include multiple AP tag subunits of EEEDDDSSS (SEQ ID NO: 60), DDDEEESSS (SEQ ID NO: 61), and SSSEEEDDD (SEQ ID NO: 62), in which three repeats of glutamic acid (E), aspartic acid (D), and serine (S), are alternatively arranged. The AP tag subunits can be directly connected to one another, or can be connected through an amino acid linker. In some embodiments, the individual AP tag subunits can be connected to one another via a two-glycine (G) residue linker. In other embodiments, residues having intrinsic flexibility similar to that of glycine can be used as a linker in place of glycine. In certain aspects, an AP tag includes an approximately equal number of each of S, E, and D residues. In embodiments where a two-glycine residue linker connects the individual AP tag subunits, the AP tag can include an approximately equal number of each of S, E, and D residues, with G residues being present in lower numbers. In some embodiments, AP tag does not form any particular secondary structure (see FIG. 4A).

In some embodiments, an AP tag can include about 5 to about 30 AP tag subunits. In some embodiments, an AP tag can include about 6 to about 27 AP tag subunits. A resulting AP tag can include, but is not limited to, from about 60 to about 300, from about 70 to about 300, from about 80 to about 300, from about 90 to about 300, from about 100 to about 300, from about 60 to about 250, from about 60 to about 200, from about 60 to about 150, from about 60 to about 100, from about 80 to about 200, from about 90 to about 200, and from about 100 to about 200 total residues. The AP tag subunits can be present in any order, so long as the AP tag does not form any secondary structure. As represented by FIG. 4A, an AP tag will generally form a random coil. Secondary structure can be well predicted using computer modeling methods, such as, for example, the PHD secondary structure prediction program (B. Rost et al., Comput Appl Biosci 10, 53-60 (1994), which is hereby incorporated by reference in its entirety). In some embodiments, the AP tag has a random coil configuration.

In certain embodiments, the AP tag can include one or more amino acids other than S, E, and D. In an AP tag including such other amino acids, the amino acids other than S, E, and D do not significantly alter the form and function of the AP tag relative to an AP tag including only S, E, and D residues. Amino acids other than S, E, and D that can be included in an AP tag that are not likely to significantly alter the form and function of the AP tag relative to an AP tag including only S, E, and D residues include glycine (G) (which as described herein, can be used as a subunit linker), and neutral residues such as, for example, alanine (A). In certain embodiments, the presence of one or more amino acids other than S, E, and D does not result in the formation of any secondary protein structure, and does not significantly affect the ability of the AP tag to improve the solubility of a target protein, including large target proteins having molecular weights of about 100 kDa or greater.

In some embodiments, AP tags can include, but are not limited to, at least one polypeptide having at least about 90%, 91%, 92%, 93%, 94%, 95%, 96%, 97%, 98%, 99%, and/or 100% sequence identity to AP100 (SEQ ID NO: 63), AP200 (SEQ ID NO: 64), AP204 (SEQ ID NO: 65), and/or AP200F (SEQ ID NO: 66). In one embodiment, the AP tag is AP200F (SEQ ID NO: 65; encoded by a polynucleotide having the sequence of SEQ ID NO: 3).

Other embodiments provide modified AP tags that do not include the AP tag subunits, but rather include about 75 to about 300 randomly or nearly randomly arranged glutamic acid (E), aspartic acid (D), and serine (S) residues. In some embodiments, the modified AP tag does not form any secondary structure. In certain embodiments, the modified AP tag can include S, E, and D residues in any ratio, and in particular embodiments, can include one or more amino acids other than S, E, and D. In embodiments where a modified AP tag includes such other amino acids, the amino acids other than S, E, and D do not significantly alter the form and function of the modified AP tag relative to a modified AP tag having only S, E, and D residues. Amino acids other than S, E, and D that can be included in a modified AP tag that are not likely to significantly alter the form and function of the modified AP tag relative to a modified AP tag including only S, E, and D residues include glycine (G), and neutral residues such as, for example, alanine. In some embodiments, the presence of one or more amino acids other than S, E, and D does not result in the formation of secondary protein structures, and does not significantly affect the ability of the modified AP tag to improve the solubility of a target protein, including large target proteins having molecular weights of about 100 kDa or greater.

Certain embodiments provide an “SED” solubility tag. In some embodiments, the SED tag can include tri-amino acid repeats of SED, EDS, DES, or any combination thereof (FIG. 4B). In particular embodiments, the SED tag can include from about 50 to about 100 tri-amino acid repeats. In certain embodiments, an SED tag can include about 65 to about 100 tri-amino acid repeats. In other embodiments, the SED tag can include about 65 to about 75 SED tri-amino acid repeats. In certain embodiments, the SED tag can include, but is not limited to, at least one polypeptide having at least about 90%, 91%, 92%, 93%, 94%, 95%, 96%, 97%, 98%, 99%, and/or 100% sequence identity to SEQ ID NO: 93, SEQ ID NO: 94, or SEQ ID NO: 95. In one embodiment, the SED tag is encoded by a polynucleotide having the sequence of SEQ ID NO: 4.

Many different combinations of tri-amino acid repeats are possible in SED tags of the embodiments herein. For example, in some embodiments SED tags can include 20, 21, 22, 23, 24, 25, 26, 27, 28, 29, 30, 31, 32, 33, 34, or 35 SED tri-amino acid repeats, followed by 5, 6, 6, 7, 8, 9, 10, 11, 12, 13, 14, or 15 EDS tri-amino acid repeats, followed by 2, 3, 4, 5, 6, 7, 8, 9, or 10 DES amino acid repeats, and ending in another 2, 3, 4, 5, 6, 7, 8, 9, or 10 SED tri-amino acid repeats. In certain embodiments, the SED tag does not form any particular secondary structure (see FIG. 4B). Methods for predicting secondary protein structure are well known in the art, such as the PHD secondary structure prediction program. In some embodiments, an SED tag including SED tri-amino acid repeats forms a random coil. An SED tag can comprise any combination of the tri-amino acid repeats of SED, EDS, and DES where the resulting SED tag can improve the solubility of a target protein, including large target proteins having molecular weights of about 100 kDa or greater.

In certain embodiments, the SED tag can include one or more amino acids outside of the tri-amino acid repeats of SED, EDS, and DES, as long as the one or more amino acids does not confer a secondary structure to the SED tag. In some embodiments, an SED tag can include tri-amino acid repeats as described above, interspersed with one or more other amino acids. The one or more other amino acids can be any amino acid, including glutamic acid (E), aspartic acid (D), and serine (S). An SED tag including 70 SED tri-amino acid repeats, for example, can be interspersed by one or more serine residues (e.g., 10×SED-SSSS-30×SED-S-15×SED-SS-15×SED). In other embodiments, the SED tag, in addition to the tri-amino acid repeats, can include any other amino acid, in any number, where the other amino acid(s) does not significantly affect the ability of the SED tag to increase the solubility of a target protein when expressed from an expression system, including large target proteins having molecular weights of about 100 kDa or greater, relative to an SED tag free of the other amino acid(s), and does not confer a secondary structure to the SED tag.

Also provided in certain embodiments are polynucleotides that encode SEP tag disclosed herein. Polynucleotides encoding the SEP tags described can be generated by any method known in the art. See, e.g., U.S. Pat. No. 8,808,989, Caruthers M H. Gene Synthesis Machines: DNA chemistry and its Uses. Science 1985; 230(4723):281-5, Carlson R, The changing economics of DNA synthesis. Nature Biotechnol. 2009; 27:1091-4, Lashkari D A, Hunicke-Smith S P, Norgren R M, Davis R W, Brennan T. An automated multiplex oligonucleotide synthesizer: development of high-throughput, low-cost DNA synthesis. Proc Natl Acad Sci USA. 1995; 92(17):7912-15, Lee C V, Snyder T_{M}, Quake S R. A Microfluidic Oligonucleotide Synthesizer. Nucleic Acids Res 2010; 38:2514-21, and Matzas M, Stahler P F, Kefer N, Siebelt N, Boisguerin V, Leonard J T, et al. Next Generation Gene Synthesis by targeted retrieval of bead-immobilized, sequence verified DNA clones from a high throughput pyrosequencing device. Nat Biotechnol. 2010; 28(12):1291-1294.

### Solubility-Enhancing-Protein Assisted Protein Expression System

Embodiments described herein also provide SEP expression vectors and expression systems. In certain embodiments, a polynucleotide having a sequence that encodes any of the SEP tags described above can be synthesized and introduced and incorporated into a vector backbone to produce a SEP expression vector. The sequences of the polynucleotides can be codon optimized for expression in a particular expression system. Expression vectors including the SEP tag-encoding polynucleotide and a polynucleotide having a sequence that encodes a target protein can be introduced into a cell of a cell expression system. Cells transfected with the expression vector can then produce soluble recombinant target protein. In some embodiments the SEP tag polynucleotide and the target protein polynucleotide are so linked that a SEP-target protein recombinant protein can be expressed from the expression vector.

In certain embodiments, polynucleotides having a SEP tag-encoding sequence can be introduced and incorporated into an expression vector backbone. Expression vector backbones can be selected dependent on the desired protein expression system. In some embodiments, expression systems can include those derived from bacteria, yeast, baculovirus/insect, mammalian, and plant cells. Each expression system can have its own unique benefits, and will each be best suited to a particular application.

Many factors are considered when selecting an appropriate expression system suitable for expressing a particular protein, including cell growth, complexity and cost of growth medium, expression levels, extracellular expression of the target recombinant protein, protein folding, N- and O-linked glycosylation, phosphorylation, acetylation, acylation, and gamma-carboxylation. In certain embodiments where the target protein is relatively large (MW of about 100 kDa or greater), an expression system having a slower cell growth rate while providing for acceptable yields and proper protein folding (e.g., the cells comprise requisite chaperone proteins) can be used. In particular embodiments, cell expression systems useful for producing large mammalian recombinant proteins can be baculovirus/insect cell and/or bacterial (e.g., E. coli) expression systems or mammalian cell expression systems. Insect cells, for example, are able to carry out more complex post-translational modifications than either bacteria or yeast, and have optimal machinery for the folding of mammalian proteins. In other embodiments, recombinant plant proteins can be similarly produced in plant cell-based expression systems.

Many expression vector backbones suitable for use in a particular expression system are known and are commercially available. Vector backbones useful in the embodiments described herein can include replication origins (e.g., pMB1, f1 Ori), antibiotics resistance (e.g., Amp R, Gen R), promoters (e.g., polh, p10), terminators, and transposition sequences (e.g., Tn7R and Tn7L). An appropriate vector backbone can be selected for any given situation. Selection of an appropriate vector backbone can depend on several factors including, but are not limited to, the particular host cell to be transformed with the expression vector and the size of the polynucleotide to be inserted into the vector. In some embodiments, a vector backbone can include, for example, one or more of: an origin or replication, a signal sequence, one or more marker genes, an enhancer element, a promoter, and a transcription termination sequence. Any expression vector backbone suitable for expressing a large target protein can be modified to include a polynucleotide encoding a SEP tag. General techniques for the manipulation of polynucleotides and vectors of interest, and cloning exogenous polynucleotides into an expression vector backbone, are known in the art. See, e.g., Allison, L. (2009). Recombinant DNA Technology and Molecular Cloning. In Fundamental Molecular Biology (p. 752). Wiley-Blackwell; Ausubel, F. M., et al., eds (2002) Short Protocols in Molecular Biology, 5th edn. John Wiley & Sons, New York; and Sambrook, J., Russell, D. W. (2001) Molecular Cloning: a Laboratory Manual, 3rd edn. Cold Spring Harbor Laboratory Press, New York.

In certain embodiments, recombinant protein expression vectors incorporating SEP tags can be generated, resulting in a Solubility-Enhancing-Protein assisted protein expression system, or SEP system. For example, polynucleotides encoding either AP or SED tags, when incorporated into a baculovirus and/or bacterial (e.g., E. coli) expression vector backbone along with polynucleotides encoding either NRPD1 or NRPD2, significantly improved the solubility of the expressed proteins, with approximately 50% of AP- or SED-tagged protein appearing in the soluble fraction. SEP systems of embodiments described herein can stabilize the target protein, enhancing its solubility without any toxic effects on the host cell (see, e.g., FIG. 1).

Expression vectors of a SEP system of the embodiments described herein can have a polynucleotide having a sequence encoding an AP or SED solubility-enhancing tag. In some embodiments, in addition to the polynucleotide sequence encoding the AP or SED solubility-enhancing tag, expression vectors of the SEP system can include one or more polynucleotides having a sequence encoding one or more of a: ribosomal binding site; linker peptide; promoter; cloning site; target protein; affinity tag; solubility-enhancing tag; yield-improving tag; and protease recognition site. Protein tags, including affinity, solubility enhancing, and yield-improving tags can have multiple effects on a recombinant protein. As such, it will be recognized that certain tags can fit into one or more of these categories.

A ribosomal binding site (RBS) is an mRNA sequence that is bound by the ribosome when initiating protein translation. Many such sites are known in the art, and can be selected for use in a particular cell expression system, including SEP systems of the present embodiments. In certain embodiments where a SEP vector to be expressed in an insect cell, the polynucleotide encoding the RBS can have the sequence of SEQ ID NO: 1. In other embodiments, the SEP vector does not encode an RBS.

In some embodiments, polynucleotides encoding linker peptides can be included in the SEP system expression vector. Polynucleotides encoding linker peptides can be provided between any two polynucleotide sequences encoding a polypeptide. For example, a linker sequence can be placed between a SEP tag-encoding polynucleotide and a multi-cloning site, between a SEP tag-encoding polynucleotide and a target protein-encoding polynucleotide, between a SEP-encoding polynucleotide and a protease recognition site and between the protease recognition site and a target protein-encoding polynucleotide, or between a SEP tag-encoding polynucleotide and any polynucleotide encoding a protein tag that is not the SEP tag-encoding polynucleotide. Linker peptides can assist in connecting two independent protein domains, forming a stable fusion protein. The length of linker peptides can vary from about 2 to about 31 amino acids, and can be optimized for a particular application so that the linker peptide does not constrain the fusion protein. Methods for designing and applying linker peptides are known in the art, for example, in Yu et al., (2015) Biotechnol Adv, January-February; 33(1):155-64 and Chen et al., (2013) Adv Drug Deliv Rev, October; 65(10):1357-69.

In other embodiments, a SEP system expression vector can include a promoter. The promoter can be any promoter capable of driving expression of the SEP tag, the target protein, or both the SEP tag and the target protein. In some embodiments, one or more promoters can be present in a SEP system expression vector. In certain embodiments, at least one promoter is operably linked to the polynucleotide encoding the SEP tag of the vector. That is, at least one promoter is linked to a polynucleotide having a sequence that encodes a SEP tag in a manner that promotes the expression of the polynucleotide encoding the SEP tag. Polynucleotide sequences which are operably linked are not necessarily physically linked directly to one another, but can be separated by intervening nucleotides which do not interfere with the operational relationship of the linked sequences. Similarly, when referring to joined polypeptide sequences, operationally linked means that the functionality of the individual joined segments are substantially identical as compared to their functionality prior to being operationally linked. For example, in some embodiments, a SEP tag can be fused to a target protein via a protease recognition site, and in the fused state, each of the SEP tag, protease recognition site, and target protein retain their individual biological activities. Suitable promoters are known in the art. In certain embodiments, a SEP expression vector can include one or both of polh and p10 promoters.

In some embodiments, a SEP system expression vector can include one or more cloning sites, or multiple cloning sites, for cloning of a target protein-encoding polynucleotide in-frame with a SEP tag-encoding polynucleotide. In certain embodiments, the SEP expression vector can include one multiple cloning site. In other embodiments, the SEP expression vector can include two multiple cloning sites. Multiple cloning sites can contain up to about 20 restriction sites, and allow for the insertion of target protein-encoding polynucleotides into the vector. Many multiple cloning sites are known in the art, and can be designed for a specific application. In certain embodiments, SEP expression vectors can include one or more multiple cloning sites such as, for example, MCS (SEQ ID NO: 23), MCS1 (SEQ ID NO: 24), and MCS2 (SEQ ID NO: 25), where the MCS and MCS2 sequences also encode a 3C protease cleavage site. In some embodiments, the 3C protease cleavage site can be omitted from MCS and MCS2. In certain embodiments, least one of the one or more cloning sites, or multiple cloning sites, can be located downstream of the SEP tag-encoding polynucleotide of the SEP expression vector.

Any protein-encoding polynucleotide can be incorporated into a SEP system expression vector as a target protein-encoding polynucleotide. In certain embodiments, the target protein to be expressed by a SEP system expression are those proteins that have proven difficult to express in other expression systems due to their size, insolubility, or both. In some embodiments, SEP system expression vectors can express proteins having a molecular weight of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater, and are difficult to express in a soluble form. Examples of target proteins include, but are not limited to, NRPD1, NRPD2, BRCA1, LRRK2, and DNA-PKcs. In some embodiments, target protein-encoding polynucleotide can be inserted at a restriction site located within a cloning site or multiple cloning site. This location results in the target protein-encoding polynucleotide to be downstream of the SEP tag-encoding polynucleotide. Expression of the target protein-encoding polynucleotide can thus be driven by the same promoter or promoters driving expression of the SEP tag-encoding polynucleotide of the SEP system expression vector.

In other embodiments, polynucleotides encoding affinity tags can be included in the SEP system expression vector. Affinity tags can aid in the purification of a target protein. Many affinity tags are known in the art, including, for example, polyhistidine, polyarginine, FLAG, hemagglutinin antigen (HA), c-myc, chitin binding protein (CBP), maltose binding protein (MBP), glutathione-S-transferase (GST), streptavidin, thioredoxin, and intein. In certain embodiments, a SEP system expression vector can include a poly(His) tag-encoding polynucleotide. When included, the encoded poly(His) tag can have about 6 to about 14 histidine residues. In one embodiment, a polynucleotide encoding a 10×His tag can be included in the SEP expression vector. Small and unlikely to affect recombinant protein function, His-tagged proteins can be purified using metal-affinity chromatography, such as a Ni2⁺ column.

In some embodiments, polynucleotides encoding known solubility-enhancing protein tags can be included in a SEP expression vector in addition to a polynucleotide encoding an AP or SED tag. Solubility-enhancing protein tags can include, for example, small ubiquitin-like modifier (SUMO), GST, MBP, N-utilization substance (NusA), thioredoxin, IgG domain B1 of Streptococcus Protein G (GB1), and HaloTag. In certain embodiments, a polynucleotide encoding a solubility-enhancing protein tag that is neither AP nor SED is included in a SEP system expression vector not for the solubility-enhancing properties of the protein it encodes, but rather for another purpose, such as yield-enhancement.

In many cases, affinity tags and/or solubility enhancing protein tags can improve recombinant protein yield. In certain embodiments, a polynucleotide encoding MBP is included in the SEP system cassette. When included in a SEP system expression vector, MBP improves the yield of AP-tagged NRPD1 and NRDP2 (see FIG. 3C, lanes 28 and 34). MBP has the same yield-improving effect on SED-tagged NRPD2 (see FIG. 3C, lane 36). Large quantities of the recombinant MBP-AP-NRDP1 (FIG. 5, lane 1), MBP-SED-NRPD1 (FIG. 5, lane 2), MBP-AP-NRDP2 (FIG. 5 lane 4), and MBP-SED-NRPD2 (FIG. 5, lane 5) proteins can be obtained in soluble forms.

In certain embodiments, any polynucleotide encoding an affinity tag, solubility-enhancing tag, or yield-enhancing tag will be located upstream of a SEP tag in the SEP system expression vector. Such tag-encoding polynucleotides can be located downstream of the one or more promoters driving expression of the SEP tag, so that the same one or more promoters drive expression of SEP tag and any other protein tags located between the promoter and the SEP tag.

Some embodiments of the SEP system expression vector can include a polynucleotide encoding a protease recognition site between the SEP tag-encoding polynucleotide and the target protein-encoding polynucleotide. Utilizing a protease recognition site can allow for the SEP tag to be separated from the expressed target protein. The removal of the SEP tag, and any other upstream tags, allows for better access to the target protein itself for further study or use, and minimizes the risk that any target protein-associated tag interferes with the target protein's structure or function. Many protease recognition sites known in the art have been recognized as being useful in the processing of recombinant fusion proteins, any of which can be incorporated into a SEP system expression vector as a protease recognition site-encoding polynucleotide. Certain embodiments can include one or more protease recognition sites including, but are not limited to, the rhinovirus 3C protease recognition site, the TEV protease recognition site, the Factor Xa protease recognition site, the thrombin protease recognition site, the enteropeptidase recognition site, the carboxypeptidase A recognition site, the carboxypeptidase B recognition site, and the DAPase recognition site.

Examples of SEP vectors that can express target proteins in their soluble form are provided in Table 1. SEP expression vectors are not limited to these examples. Based on the present disclosure, those of skill in the art can design additional SEP vectors. A schematic representation of SEP expression vector examples is provided in FIG. 2. In some cases, pFastBac1 vector (Invitrogen) can be utilized as a starting template for vectors, such as those provided in Table 1. Starting template can be modified as outlined in the methods section. Based on the present disclosure, one of skill in the art can substitute the protein tags and/or protease recognition site of the SEP vectors provided in the examples or utilize different base vectors without departing from the essential scope of the SEP vectors described herein. Further, SEP vectors having minor modifications relative to those provided in Table 1 are contemplated, and can include any SEP vector having a sequence identity of at least about 90%, 91%, 92%, 93%, 94%, 95%, 96%, 97%, 98%, 99%, and/or 100% to that of one of the examples.

Also provided in certain embodiments are nucleic acid cassettes for insertion of DNA encoding an SEP tag into any recombinant vector system. In some embodiments, a nucleic acid cassette can be made up of a polynucleotide encoding a SEP tag such as, for example, at least one sequence having at least about 90%, 91%, 92%, 93%, 94%, 95%, 96%, 97%, 98%, 99%, and/or 100% sequence identity to SEQ ID NO: 3 (AP tag) or one of SEQ ID NOs: 93-95 (SED tags). In some embodiments, the nucleic acid cassette can further include one or more polynucleotide having a sequence encoding one or more of: ribosomal binding site: linker peptide; promoter; cloning site; target protein; affinity tag; solubility-enhancing tag; yield-improving tag; and protease recognition site. Such polynucleotides are disclosed and discussed above. The nucleic acid cassette can also comprise a gene encoding a target protein.

A nucleic acid cassette can be inserted into any suitable recombinant vector system to produce a target protein in soluble form.

TABLE 1
Representative SEP vectors useful for expressing a target
protein in its soluble form.

| | | Number of | SEQ |
|---|---|---|---|
| | Tag and SEP | Multiple Cloning | ID |
| Vector | Description | Sites | NO: |
| pSEP1Sb | RBS-10xHis-MBP-AP- | 1 | 16 |
| | 3C | | |
| pSEP2Sb | RBS-10xHis-MBP-SED- | 1 | 17 |
| | 3C | | |
| pSEP3Sb | RBS-10xHis-SUMO-AP- | 1 | 18 |
| | 3C | | |
| pSEP4Sb | RBS-10xHis-SUMO- | 1 | 19 |
| | SED-3C | | |
| pSEP1(Dual)b | RBS-10xHis-MBP-AP- | 2 | 21 |
| | 3C | | |
| pSEP2(Dual)b | RBS-10xHis-MBP-SED- | 2 | 22 |
| | 3C | | |
| pSEP1Sa | 10xHis-MBP-AP-3C | 1 | 76 |
| pSEP2Sa | 10xHis-MBP-SED-3C | 1 | 77 |
| pSEP3Sa | 10xHis-SUMO-AP-3C | 1 | 78 |
| pSEP4Sa | 10xHis-SUMO-SED-3C | 1 | 79 |
| pSEP5Sa | MBP-AP-3C | 1 | 80 |
| pSEP6Sa | MBP-SED-3C | 1 | 81 |
| pSEP1(Dual)a | 10xHis-MBP-AP-3C | 2 | 83 |
| pSEP2(Dual)a | 10xHis-MBP-SED-3C | 2 | 84 |
| pSEP3(Dual)a | 10xHis-SUMO-AP-3C | 2 | 85 |
| pSEP4(Dual)a | 10xHis-SUMO-SED-3C | 2 | 86 |
| pSEP5(Dual)a | MBP-AP-3C | 2 | 87 |
| pSEP6(Dual)a | MBP-SED-3C | 2 | 88 |

Examples of embodiments of nucleic acid cassettes are provided in Table 2, and it is intended that nucleic acid cassettes not be limited to these examples. A schematic representation of several of the nucleic acid cassette examples can be found in FIG. 1A, where the cassettes further include a polynucleotide encoding a target protein. Based on the present disclosure, one of skill in the art can substitute a nucleic acid sequence encoding a protein tags and/or protease recognition site of the nucleic acid cassette provided in the examples without departing from the essential scope of the nucleic acid cassettes described herein. Further, nucleic acid cassettes having minor modifications to those provided in Table 2 are contemplated, and can include any nucleic acid cassette having at least one sequence having at least about 90%, 91%, 92%, 93%, 94%, 95%, 96%, 97%, 98%, 99%, and/or 100% sequence identity to that of one of the examples.

TABLE 2
Representative nucleic acid cassettes.

| | SEQ |
|---|---|
| | ID |
| Description | NO: |
| RBS-10xHis-MBP-AP-3C | 5 |
| RBS-10xHis-MBP-SED- | 6 |
| 3C | |
| RBS-10xHis-SUMO-AP- | 10 |
| 3C | |
| RBS-10xHis-SUMO- | 11 |
| SED-3C | |
| 10xHis-MBP-AP-3C | 67 |
| 10xHis-MBP-SED-3C | 68 |
| 10xHis-SUMO-AP-3C | 69 |
| 10xHis-SUMO-SED-3C | 70 |
| MBP-AP-3C | 71 |
| MBP-SED-3C | 72 |
| SUMO-AP-3C | 73 |
| SUMO-SED-3C | 74 |

In some embodiments, the SEP system described herein can produce large target proteins in a soluble form, where the target protein had previously been difficult to produce in any appreciable quantity. In other embodiments, recombinant fusion proteins including a SEP tag described herein are also provided.

In some embodiments, a recombinant fusion protein includes a SEP tag. The SEP tag can be fused directly or indirectly to a target protein. The resulting fusion protein can be expressed and recoverable in a soluble form. In embodiments where the SEP tag is indirectly fused to a target protein, one or more proteins including, but are not limited to, protease recognition sites and linker peptide, can be located between the SEP tag and the target protein. Recombinant fusion proteins can further comprise one or more additional protein tags, such as affinity tags, solubility-enhancing tags, and yield-improving tags. In certain embodiments, for example, a recombinant fusion protein can comprise a polyhistidine tag, an MBP tag, and a protease recognition site in addition to the SEP tag and target protein. Such recombinant fusion proteins can be easily purified due to the presence of the polyhistidine tag, and are expressed at improved yields at least in part due to the MBP tag, and following purification, the target protein can be separated from the other elements of the recombinant fusion protein by cleavage at the protease recognition site. Those of skill in the art will recognize that a similar strategy can be pursued using other protein tags known in the art, as described herein.

Also provided in embodiments herein are methods for expressing and producing a recombinant fusion protein. The methods enable the expression and isolation of a target protein in its soluble form where the target protein is generally considered difficult to express without the benefit of the present disclosure. Target proteins can be large proteins having molecular weights of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater. Examples of recombinant fusion proteins including a target protein producible by the methods provided herein include, but are not limited to, the target proteins NRPD1, NRPD2, BRCA1, LRRK2, DNA-PKcs, MED12, RRM3, mTOR, LYP, and CTCR. Many other difficult to express target proteins can be expressed and produced as a recombinant fusion protein by the methods herein.

Particular target proteins may be best expressed using either the AP tag or the SED tag, as recombinant target protein solubility or yield may differ depending on the fused SEP tag. Soluble recombinant target protein expression can easily be optimized by determining which SEP tag provides better yields of soluble protein.

In some embodiments, recombinant fusion protein including at least a SEP tag and a target protein can be produced by providing a SEP expression vector encoding the recombinant fusion protein and expressing the fusion protein from the vector (see FIG. 1B). Expression of the fusion protein from the vector can result from introducing the SEP expression vector encoding the recombinant fusion protein into an appropriate cell capable of expressing the heterologous fusion protein. The propriety of a particular cell type for use in expressing the recombinant fusion protein can depend on many factors, such as the backbone of the SEP expression vector, cell growth rates, expression levels, extracellular expression of the target recombinant protein, protein folding, and protein processing, including O-linked glycosylation, phosphorylation, acetylation, acylation, and gamma-carboxylation. Any SEP vector described herein can be utilized to express and produce a recombinant fusion protein employing conventional molecular biology, microbiology, and recombinant DNA techniques. See, e.g., Allison, L. (2009). Recombinant DNA Technology and Molecular Cloning. In Fundamental Molecular Biology (p. 752). Wiley-Blackwell; Ausubel, F. M., et al., eds (2002) Short Protocols in Molecular Biology, 5th edn. John Wiley & Sons, New York; and Sambrook, J., Russell, D. W. (2001) Molecular Cloning: a Laboratory Manual, 3rd edn. Cold Spring Harbor Laboratory Press, New York.

In some embodiments, the expressed recombinant fusion protein including at least a SEP tag and a target protein can be purified by standard methods. Purification can involve, for example, column chromatography targeting the SEP tag, the target protein, or another protein tag of the recombinant fusion protein. In embodiments where the recombinant fusion protein includes a polyhistidine tag, nickel or cobalt-based affinity chromatography columns can be used. In other embodiments, purification steps can include secondary chromatographic techniques to minimize impurities.

Soluble target protein can be separated from the remainder of the fusion protein (see FIG. 1C). This can be facilitated by including a protease recognition site in the recombinant fusion protein. In embodiments where the protease recognition site is a 3C recognition site, for example, rhinovirus 3C protease can be used to separate the soluble target protein from the remainder of the fusion protein (see FIG. 1C).

Yet other embodiments provide kits including a SEP expression vector or cassette encoding a SEP tag described herein. The SEP expression vector of the kit can, for example, encode a fusion protein including an affinity tag, yield-improving tag, a SEP tag, and a protease recognition site. The SEP vector of the kit can include at least one cloning site or multiple cloning site to allow a user to insert one or more target protein-encoding polynucleotides into the SEP vector, where at least one target protein-encoding sequence is linked to a SEP tag-encoding sequence to allow for the expression of a SEP tag-target protein fusion protein. In some embodiments, the kit may further comprise an appropriate affinity chromatography column and associated buffers capable of purifying the fusion protein encoded by the SEP vector, an appropriate protease for cleaving a protease recognition site encoded by the SEP vector to allow for separation of the target protein from the protein tags encoded by the SEP vector, or both.

A first embodiment includes an expression vector comprising a vector backbone, at least one polynucleotide sequence encoding a solubility-enhancing polypeptide comprising an AP tag and/or an SED tag.

A second embodiment includes the expression vector according to the first embodiment, wherein the AP tag comprises at least five AP tag subunits chosen from EEEDDDSSS (SEQ ID NO: 60), DDDEEESSS (SEQ ID NO: 61), and SSSEEEDDD (SEQ ID NO: 62), wherein the AP tag comprises from about 6 to about 35, from about 6 to about 30, from about 6 to about 29, from about 6 to about 28, from about 6 to about 27, from about 6 to about 26, from about 6 to about 25, from about 7 to about 35, from about 8 to about 35, from about 9 to about 35, from about 10 to about 35 of the AP tag subunits, or 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 AP tag subunits.

A third embodiment includes the expression vector according to the second embodiment, wherein the AP tag subunits of EEEDDDSSS (SEQ ID NO: 60), DDDEEESSS (SEQ ID NO: 61), and SSSEEEDDD (SEQ ID NO: 62) are present in approximately equal numbers.

A fourth embodiment includes the expression vector according to any one of the second or third embodiments, wherein the AP tag subunits are connected via at least two glycine residues.

A fifth embodiment includes the expression vector according to any one of the second to the fourth embodiments, wherein one or more residues from the AP tag subunits is modified to avoid formation of a secondary structure.

A sixth embodiment includes the expression vector according to any one of the first to the fifth embodiments, wherein the AP tag comprises no amino acid residues other than serine, glutamic acid, aspartic acid, and glycine.

A seventh embodiment includes the expression vector according to any one of the first to the sixth embodiments, wherein the AP tag comprises at least one polypeptide having at least about 90%, 91%, 92%, 93%, 94%, 95%, 96%, 97%, 98%, 99%, and/or 100% sequence identity to SEQ ID NO: 66.

An eighth embodiment includes the expression vector according to any one of the first to the seventh embodiments, wherein the expression vector comprises the SED tag, wherein the SED tag comprises at least 20 three-amino acid repeats chosen from having serine-glutamic acid-aspartic acid (SED), glutamic acid-aspartic acid-serine (EDS), and aspartic acid-serine-glutamic acid (DSE). Consistent with these embodiments, the SED tag comprises about 20, about 25, about 30, about 35, about 40, about 45, about 50, about 55, about 60, and/or about 65 of the three-amino acid repeats (SED, EDS, DSE), wherein the three amino acid repeats are each present in a predetermined number. For examples, 20 SED repeats, 20 EDS, repeats, and 20 DSE repeats; 30 SED repeats, 30 EDS repeats, 0 DSE repeats; 60 SED repeats, 0 EDS repeats, 0 DSE repeats The various repeats can be interspersed amongst each other.

A ninth embodiment includes the expression vector according to the first to the eighth embodiments, wherein the SED tag comprises at least one polypeptide having at least about 90%, 91%, 92%, 93%, 94%, 95%, 96%, 97%, 98%, 99%, and/or 100% sequence identity to SEQ ID NO: 4.

A tenth embodiment includes the expression vector according to the first to the ninth embodiments, wherein the at least one polynucleotide is operably linked to a promoter sequence.

An eleventh embodiment includes the expression vector according to any one of the first to the tenth embodiments, further comprising a multiple cloning site downstream of the at least one polynucleotide encoding the solubility-enhancing polypeptide.

A twelfth embodiment includes the expression vector according to any one of the first to the eleventh embodiments, wherein the at least one polynucleotide encoding the solubility-enhancing polypeptide is operably linked to the at least one polynucleotide encoding a target protein.

A thirteenth embodiment includes the expression vector according to any one of the first to the twelfth embodiments, wherein the target protein has a size of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater.

A fourteenth embodiment includes the expression vector according to any one of the first to the thirteenth embodiments, further comprising at least one polynucleotide encoding at least one protein tag upstream of the at least one polynucleotide encoding the solubility-enhancing polypeptide.

A fifteenth embodiment includes the expression vector according to the fourteenth embodiment, wherein the at least one protein tag comprises an affinity protein tag, a solubility-enhancing protein tag, and/or a yield-improving protein tag.

A sixteenth embodiment includes the expression vector according to the fourteenth and the fifteenth embodiments, wherein the at least one protein tag comprises a His tag and/or a maltose-binding protein (MBP) tag. Consistent with these embodiments, the at least one protein tag comprises the His tag and the MBP tag, wherein the at least one polynucleotide encoding the His tag is upstream of the at least one polynucleotide encoding the MBP tag.

A seventeenth embodiment includes the expression vector according to the fourteenth to the sixteenth embodiments, wherein the His tag comprises about 5, 6, 7, 8, 9, 10, 11, 12, 13, 14, 15, 16, 18, 19, or 20 histidine residues.

An eighteenth embodiment includes the expression vector according to any one of the fourteenth to the seventeenth embodiments, wherein the expression vector comprises two or more polynucleotides encoding two or more protein tags, wherein the two or more polynucleotides encoding two or more protein tags are separated by a polynucleotide encoding a linker peptide.

A nineteenth embodiment includes the expression vector according to any one of the first to the eighteenth embodiments, further comprising at least one polynucleotide encoding at least one protease recognition site. Consistent with these embodiments, the at least one polynucleotide encoding at least one protease recognition site is downstream of the at least one polynucleotide encoding the solubility-enhancing polypeptide and/or the at least one polynucleotide encoding the at least one protein tag, upstream of the at least one polynucleotide encoding the solubility-enhancing polypeptide and/or the at least one polynucleotide encoding the at least one protein tag, and/or in between the at least one polynucleotide encoding the solubility-enhancing polypeptide and the at least one polynucleotide encoding the at least one protein tag.

A twentieth embodiment includes the expression vector according to the nineteenth embodiments, wherein the at least one protease recognition site is an HRV 3C protease cleavage sequence.

A twenty first embodiment includes the expression vector according to any one of the first to the twentieth embodiments, wherein the at least one polynucleotide encoding the at least one protein solubility-enhancing polypeptide is operably linked to at least one polynucleotide encoding at least one target protein and the protease recognition sequence is in between the at least one polynucleotide encoding the at least one protein solubility-enhancing polypeptide and the at least one polynucleotide encoding the at least one target protein.

A twenty second embodiment includes the expression vector according to the first to the twenty first embodiments, further comprising at least two multiple cloning sites.

A twenty third embodiment includes the expression vector according to any one of the first to the twenty second embodiments, wherein the vector is a mammalian expression vector, a bacterial expression vector, and/or baculovirus expression vector.

A twenty fourth embodiment includes the expression vector according to any one of the first to the twenty third embodiments, wherein the expression vector comprises a polynucleotide having a nucleic acid sequence having at least about 90%, 91%, 92%, 93%, 94%, 95%, 96%, 97%, 98%, 99%, and/or 100% sequence identity to any one of SEQ ID NO: 16, SEQ ID NO: 17, SEQ ID NO: 18, SEQ ID NO: 19, SEQ ID NO: 21, SEQ ID NO: 22; SEQ ID NO: 76; SEQ ID NO: 77; SEQ ID NO: 78; SEQ ID NO: 79; SEQ ID NO: 80; SEQ ID NO: 81; SEQ ID NO: 83; SEQ ID NO: 84; SEQ ID NO: 85; SEQ ID NO: 86; SEQ ID NO: 87; and SEQ ID NO: 88.

A twenty fifth embodiment includes the expression vector according to any one of the first to the twenty fourth embodiments, wherein the at least one target protein comprises at least one protein that has low solubility or is insoluble when expressed in other expression systems.

A twenty sixth embodiment includes the expression vector according to any one of the first to the twenty fifth embodiments, wherein the at least one target protein comprises NRPD1/2, BRCA1, LRRK2, DNA-PKcs, MED12, RRM3, mTOR, LYP, RPS5, or CTCF.

A twenty seventh embodiment includes a method of expressing a target protein in a solution, comprising providing an expression vector according to any one of the first to the twenty sixth embodiments, wherein the expression vector comprises a polynucleotide encoding a target protein; and expressing the target protein from the expression vector.

A twenty eighth embodiment includes the method according to the twenty seventh embodiment, wherein the expression vector comprises a multiple cloning site downstream of the at least one polynucleotide encoding a solubility-enhancing polypeptide and the polynucleotide encoding the at least one target protein is inserted at the multiple cloning site.

A twenty ninth embodiment includes the method according to any one of the twenty seventh and the twenty eighth embodiments, wherein the at least one target protein has a size of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater.

A thirtieth embodiment includes the method according to any one of the twenty seventh to the twenty ninth embodiments, wherein the at least one target protein comprises at least one sequence comprising NRPD1/2, BRCA1, LRRK2, DNA-PKcs, MED12, RRM3, mTOR, LYP, RPS5, and/or CTCF.

A thirty first embodiment includes the method according to any one of the twenty seventh to the thirtieth embodiments, wherein the target protein is expressed in a recombinant host cell.

A thirty second embodiment includes the method according to any one of the twenty seventh to the thirty first embodiments, further comprising isolating and/or purifying the expressed target protein. Consistent with these embodiments, the expressed target protein is fused or otherwise connected to the at least one solubility-enhancing polypeptide.

A thirty third embodiment includes the method according to any one of the twenty seventh to the thirty second embodiments, the method comprises separating the expressed target protein from the solubility-enhancing polypeptide.

A thirty fourth embodiment includes the method according to any one of the twenty seventh to the thirty third embodiments, wherein the at least one solubility-enhancing polypeptide is removed from the expressed target protein by adding at least one protease. Consistent with these embodiments, the cleavage occurs at a protease recognition sequence.

A thirty fifth embodiment includes a kit comprising an expression vector encoding an AP tag and/or an SED tag, and at least one cloning site suitable for cloning a polynucleotide encoding at least one target protein.

A thirty sixth embodiment includes the kit according to the thirty fifth embodiment, wherein the target protein has a size of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater.

A thirty seventh embodiment includes the kit according to the thirty fifth and the thirty sixth embodiments, wherein the expression vector further encodes an affinity protein tag, a yield-enhancing protein tag, or both an affinity protein tag and a yield-enhancing protein tag.

A thirty eighth embodiment includes the kit according to the thirty fifth to the thirty seventh embodiments, wherein the kit further comprises an affinity chromatography column and at least one buffer for purifying an affinity protein-tagged target protein.

A thirty ninth embodiment includes the kit according to the thirty fifth to the thirty eighth embodiments, wherein the expression vector further encodes a protease recognition site.

A fortieth embodiment includes the kit according to the thirty fifth to the thirty ninth embodiments, further comprising a protease for cleaving the target protein from the AP tag and/or the SED tag.

A forty first embodiment includes the kit according to the thirty fifth to the fortieth embodiments, wherein the cloning site is a multiple cloning site.

A forty second embodiment includes a recombinant protein comprising at least one target protein and at least one at least one solubility-enhancing polypeptide.

A forty third embodiment includes the recombinant protein according to the forty second embodiment, wherein the at least one target protein has a size of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, or greater.

A forty fourth embodiment includes the recombinant protein according to the forty second and the forty third embodiments, wherein the recombinant protein is produced using the expression vector according to any one of the first to the twenty sixth embodiments.

A forty fifth embodiment includes the recombinant protein according to the forty second to the forty fourth embodiments, wherein the at least one target protein comprises at least one protein that has low solubility or is insoluble when expressed in standard expression systems.

A forty sixth embodiment includes the recombinant protein according to the forty second to the forty fifth embodiments, wherein the at least one solubility-enhancing polypeptide comprises an AP tag and/or an SED tag.

A forty seventh embodiment includes the recombinant protein according to the forty second to the forty sixth embodiments, wherein the AP tag comprises at least five AP tag subunits chosen from EEEDDDSSS (SEQ ID NO: 60), DDDEEESSS (SEQ ID NO: 61), and SSSEEEDDD (SEQ ID NO: 62), wherein the AP tag comprises from about 6 to about 35, from about 6 to about 30, from about 6 to about 29, from about 6 to about 28, from about 6 to about 27, from about 6 to about 26, from about 6 to about 25, from about 7 to about 35, from about 8 to about 35, from about 9 to about 35, from about 10 to about 35 of the AP tag subunits, or 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 AP tag subunits.

A forty eighth embodiment includes the recombinant protein according to the forty second to the forty seventh embodiments, wherein the AP tag subunits of EEEDDDSSS (SEQ ID NO: 60), DDDEEESSS (SEQ ID NO: 61), and SSSEEEDDD (SEQ ID NO: 62) are present in approximately equal numbers.

A forty ninth embodiment includes the recombinant protein according to the forty second to the forty eighth embodiments, wherein the AP tag subunits are connected via at least two glycine residues.

A fiftieth embodiment includes the recombinant protein according to the forty second to the forty ninth embodiments, wherein one or more residues from the AP tag subunits is modified to avoid formation of a secondary structure.

A fifty first embodiment includes the recombinant protein according to the forty second to the fiftieth embodiments, wherein the AP tag comprises no amino acid residues other than serine, glutamic acid, aspartic acid, and glycine.

A fifty second embodiment includes the recombinant protein according to the forty second to the fifty first embodiments, wherein the AP tag comprises at least one polypeptide having at least about 90%, 91%, 92%, 93%, 94%, 95%, 96%, 97%, 98%, 99%, and/or 100% sequence identity to SEQ ID NO: 66.

A fifty third embodiment includes the recombinant protein according to the forty second to the fifty second embodiments, wherein the recombinant protein comprises the SED tag, wherein the SED tag comprises at least 20 three-amino acid repeats chosen from having serine-glutamic acid-aspartic acid (SED), glutamic acid-aspartic acid-serine (EDS), and aspartic acid-serine-glutamic acid (DSE). Consistent with these embodiments, the SED tag comprises about 20, about 25, about 30, about 35, about 40, about 45, about 50, about 55, about 60, and/or about 65 of the three-amino acid repeats (SED, EDS, DSE), wherein the three amino acid repeats are each present in a predetermined number. For examples, 20 SED repeats, 20 EDS, repeats, and 20 DSE repeats; 30 SED repeats, 30 EDS repeats, 0 DSE repeats; 60 SED repeats, 0 EDS repeats, 0 DSE repeats The various repeats can be interspersed amongst each other.

A fifty fourth embodiment includes the recombinant protein according to the forty second to the fifty third embodiments, wherein the SED tag comprises at least one polypeptide having at least about 90%, 91%, 92%, 93%, 94%, 95%, 96%, 97%, 98%, 99%, and/or 100% sequence identity to SEQ ID NO: 4.

A fifty fifth embodiment includes the recombinant protein according to the forty second to the fifty fourth embodiments, wherein the recombinant protein further comprises at least one an affinity protein tag, at least one yield-improving protein tag, and/or at least one protease cleavage site.

A fifty sixth embodiment includes the recombinant protein according to the forty second to the fifty fifth embodiments, wherein the at least one affinity protein tag comprises a His tag and/or a maltose-binding protein (MBP) tag. In some embodiments, the at least one protein tag comprises the His tag and the MBP tag, wherein the at least one polynucleotide encoding the His tag is upstream of the at least one polynucleotide encoding the MBP tag.

A fifty seventh embodiment includes the recombinant protein according to the forty second to the fifty sixth embodiments, wherein the recombinant protein is soluble.

A fifty eighth embodiment includes the recombinant protein according to the forty second to the fifty seventh embodiments, wherein the at least one target protein comprises NRPD1/2, BRCA1, LRRK2, DNA-PKcs, MED12, RRM3, mTOR, LYP, RPS5, or CTCF.

### Examples

The materials, methods, and embodiments described herein are further defined in the following Examples. Certain embodiments of the present disclosure are defined in the Examples herein. It should be understood that these Examples, while indicating certain embodiments of the disclosure, are given by way of illustration only. From the discussion herein and these Examples, one skilled in the art can ascertain the essential characteristics of this disclosure and without departing from the spirit and scope thereof, can make various changes and modifications of the disclosure to adapt it to various usages and conditions.

### Example 1. Computation-Based Design of Solubility-Enhancing-Protein Tags AP and SED

In one exemplary method, relatively short engineered polypeptides capable of increasing overall solubility of large proteins expressed in an expression system were designed. The overall design concept was as follows: (i) solubility enhancing polypeptides (SEPs) could be generated using repetitive sequence of glutamic acid (E), aspartic acid (D), and serine (S), and (ii) engineered SEPs would not form a structure (random coil), as predicted by PHD secondary structure prediction program (B. Rost et al, Comput Appl Biosci 10, 53-60 (1994), which is hereby incorporated by reference in its entirety).

Two artificial polypeptides were designed: one termed “Acid Patch” (AP) the other termed “SED” tag. Computational analysis was performed using the program PROSO II (3), which determined that the length of AP and SED tags can be about 100 to about 200 residues in length to provide enough presumed solubilization power.

The AP tag AP100 (SEQ ID NO: 63) included 9 AP tag subunits linked to one another via a two-glycine residue linker, with three glycine residues at the terminal end. The resulting AP tag had 100 residues. Other AP tags having a similar length can be generated by rearranging the order of the AP tag subunits of AP100.

The AP tag AP200 (SEQ ID NO: 64) included two repeats of AP100. The resulting tag had 200 residues, and similarly to AP100, an AP tag having a similar length to AP200 can be generated by rearranging the order of the AP tag subunits.

Upon analyzing AP200 using the PHD secondary structure prediction program, four regions of the protein were predicted to form either α-helix or β-sheet secondary structures. Because of this, four glycine residues were inserted in the structure-forming regions to disrupt any potential structure formation. The result was AP204 (SEQ ID NO: 65). However, the PHD secondary structure prediction program predicted two regions of the peptide would form either α-helix or β-sheet secondary structures.

The amino acid sequence of AP204 was modified to disrupt any potential structure forming regions, with each modification being evaluated using the PHD secondary structure prediction program. The modifications resulted in AP200F (final) (SEQ ID NOs:3 (nucleic acid) and 66 (amino acid)). AP200F included 53 aspartic acid residues (26.5% of the 200 total residues), 46 glutamic acid residues (23%), 56 serine residues (56%), and 45 glycine residues (22.5%).

SED tags generally included repetitive sequence of S, E, and D (e.g., SEQ ID NO: 4; FIG. 4B).

### Example 2. Determination of the Length of the AP and SED Tags

In another exemplary method, various lengths of the AP and SED tags were determined. The predicted lengths of the SED and AP tags was based on the solubility prediction resulting from combining NRPD1 or NRPD2 protein sequences with the SEP tag. SED and AP tags with lengths of 50, 100, 150, or 200 amino acids were computationally generated, and fused to the N-terminus of the NRPD1 or NRPD2 protein sequences. Solubility of each fusion protein was calculated using the PROSO II program (a sequence-based PROtein SOlubility evaluator) (P. Smialowksi et al., FEBS J 279, 2192-2200 (2012), which is hereby incorporated by reference in its entirety). While non-SEP tagged versions of NRPD1 and NRPD2, and those fused with AP or SDE tags with lengths of 50 residues (termed AP50 SED50), were predicted to be insoluble (Table 3), NRPD1 fused with an AP tag of 100 residues or greater (AP100), or an SED tag with 150 residues or greater (SED150) were predicted to be soluble. NRPD2 fused with AP150 or SED200 were predicted to be soluble. Based on this computational analysis, the lengths of SEP tags that can enhance protein solubility were determined be about 100 residues or greater for the AP tag, and about 150 or greater for the SED tag, although the tag length can depend on the tag and the fused protein.

TABLE 3
Solubility prediction for SEP tags of various lengths
fused to either NRPD1 or NRPD2.

| NRPD1 | SED Solubility | AP solubility |
|---|---|---|
| Non tag | insoluble; 0.483 | |
| 50 a.a. | insoluble; 0.553 | insoluble; 0.578 |
| 100 a.a. | insoluble; 0.598 | soluble; 0.615 |
| 150 a.a. | soluble; 0.636 | soluble; 0.658 |
| 200 a.a. | soluble; 0.667 | soluble; 0.690 |
| NRPD2 | SED Solubility | AP solubility |
| Non tag | insoluble; 0.382 | |
| 50 a.a. | insoluble; 0.478 | insoluble; 0.529 |
| 100 a.a. | insoluble; 0.538 | insoluble; 0.579 |
| 150 a.a. | insoluble; 0.588 | soluble; 0.637 |
| 200 a.a. | soluble; 0.629 | soluble; 0.678 |

### Example 3. Construction of pSEP Vectors—pSEP Single and pSEP Dual

In another exemplary method, the pSEP Single vectors pSEP0Sb (SEQ ID NO: 15), pSEP1Sb (SEQ ID NO: 16), and pSEP2Sb (SEQ ID NO: 17), were generated using pFastBac1 (Invitrogen) as a starting template. The pUC1 origin of pFastBac1 was replaced with pMB1 origin from pRS322 vector purchased from Addgene. Replication origin was PCR-amplified using the primers rep_pBR322_F (SEQ ID NO: 29) and rep_pBR322_R (SEQ ID NO: 30). The vector pFastBac1 was PCR amplified by primers pFAST_rep_F (SEQ ID NO: 31) and pFAST_rep_R (SEQ ID NO: 32), using PrimeSTAR GXL DNA polymerse (Takara Co). The PCR products were used to remove pUC origin sequence from pFastBac, and replaced with pMB1 origin sequence by SLIC method (M. Z. Li and S. J. Elledge, Nat Methods 4, 251-256 (2007), which is hereby incorporated by reference in its entirety), yielding a pFastBac-MB1 vector. The pSEP0Sb (SEQ ID NO: 15) vector was generated by inserting DNA sequence encoding 10λHis tag (SEQ ID NO: 2) into pFastBac-MB1 vector by SLIC method using the primers, Pre_His_F2 (SEQ ID NO: 33) and MC2_vec_RBS_His_R (SEQ ID NO: 33).

The pSEP Single vectors pSEP0Sa (SEQ ID NO: 75), pSEP1Sa (SEQ ID NO: 76), and pSEP2a (SEQ ID NO: 77), were similarly generated using pFastBac1 (Invitrogen) as a starting template, but using the original pFastBac1 pUC1 origin.

DNAs encoding 10×His-Maltose-Binding Protein (MBP)-3C protease site-AP tag or -SED tag were synthesized by GenScript. All DNA sequences were codon optimized for expression in insect cells for use in a baculovirus/insect cell expression system. For generating pSEP1 Single b (pSEP1Sb; SEQ ID NO: 16) and pSEP2 Single b (pSEP2Sb; SEQ ID NO: 17), DNA sequence encoding MBP (SEQ ID NO: 7) was PCR amplified using the primers, FAP_RBS_vec_F (SEQ ID NO: 35) and MBP_R (SEQ ID NO: 36). DNA encoding AP (SEQ ID NO: 3) or SED (SEQ ID NO: 4) tag was PCR amplified using the primers, MBP_CT_F (SEQ ID NO: 37) and 3C_rev (SEQ ID NO: 38) primers. The primers, MC2opn_BamF (SEQ ID NO: 39) and MC2opn_Bam_R (SEQ ID NO: 40) were used to PCR amplify pFastBac-MB1 vector. The three PCR products corresponding to (i) 10×His-MPB-3C protease site, (ii) AP tag or SED tag, and (iii) pFastBac-MB1 vector backbone, were assembled by SLIC method (4), yielding pSEP1Sb (10×His-MBP-AP; SEQ ID NO16) or pSEP2Sb (10×His-MBP-SED; SEQ ID NO: 17) vectors. The pSEP0(Dual)b (SEQ ID NO: 20), pSEP1(Dual)b (SEQ ID NO: 21), and pSEP2(Dual)b (SEQ ID NO: 22)b (FIG. 2) vectors were generated from pFastBac Dual as a template, using the same strategy described above.

pSEP1 Single a (pSEP1Sa; SEQ ID NO: 76), and pSEP2 Single a (pSEP2Sa; SEQ ID NO: 77) were generated similarly, except DNA sequence encoding MBP (SEQ ID NO: 7) was PCR amplified using the primers SEP_vec_F (SEQ ID NO: 91) and MBP_R (SEQ ID NO: 36). The pSEP0(Dual)a (SEQ ID NO: 82), pSEP1(Dual)a (SEQ ID NO: 83), and pSEP2(Dual)a (SEQ ID NO: 84) vectors were generated from pFastBac1 Dual as a template, using the same strategy described above.

### Example 4. Construction of a SUMO Version of the pSEP Vectors

In another exemplary method, 10×His-SUMO tag was gene synthesized, and cloned into pUC57 vector by GenScript. For generating a SUMO-AP version of the vector (pSEP3S; SEQ ID NO: 18), DNA encoding SUMO tag was PCR amplified using the primers, SUMO_His_F (SEQ ID NO: 42) and SUMO_AP_R (SEQ ID NO: 43). The pSEP1S vector was PCR amplified using the primers, His_ATG_R (SEQ ID NO: 41) and SUMO_AP_F (SEQ ID NO: 42). The two PCR products were used to replace MBP sequence with those of SUMO by SLIC method, yielding pSEP3S vector (SEQ ID NO: 18).

For generating SUMO-SED version of the vector (pSEP4S; SEQ ID NO: 19), the same approach was taken, using primers SUMO_His_F (SEQ ID NO: 42) and SUMO_SED_R (SEQ ID NO: 43) for the insert, and the primers His_ATG_R (SEQ ID NO: 41) and SED F (SEQ ID NO: 45) for the vector. The two PCR products were used to replace MBP sequence with those of SUMO by SLIC method, yielding pSEP4S vector (SEQ ID NO: 19).

### Example 5. Construction of Non-10×His Tag Version (MBP-AP and MBP-SED) of the pSEPa Vectors

In other exemplary methods, non-10×His tagged versions of pSEPa vectors were constructed. To construct pSEP5Sa (MBP-AP; SEQ ID NO: 80) and pSEP6Sa (MBP-SED; SEQ ID NO: 81) vectors, DNA sequence corresponding to 10×His tag was removed from pSEP1a and pSEP2a vectors by the SLIC method using the primers Remv_His_F (SEQ ID NO: 89) and Remv_His_R (SEQ ID NO: 90). The pSEP5(Dual)a (SEQ ID NO: 87), and pSEP6(Dual)a (SEQ ID NO: 88), vectors were generated from pSEP1(Dual)a (SEQ ID NO: 83), or pSEP2(Dual)a (SEQ ID NO: 84) as a template, using the same strategy with the same primers (Remv_His_F and Remv_His_R) described above.

### Example 6. Construction of SEP Transfer Vectors for NRPD1, NRPD2, hLRRK2, DNA-PK Catalytic Subunit, Yeast Med12, CTCF, hBRCA1, CTCF, Lymphoid-Specific Protein Tyrosine Phosphatase (Lyp) and Yeast Rrm3

In other exemplary methods, SEP transfer vectors were constructed. NRPD1, NRPD2, and human LRRK2 were gene synthesized by GenScript. Each gene sequence was codon optimized for expression in the baculovirus/insect cell expression system, as well as for removing unwanted restriction enzyme sites including BamHI, HindIII, NruI, SpeI, SmaI, and SphI. Synthesized genes were cloned into pUC57 vector followed by direct sub-cloning of the synthesized DNA into BamHI and HindIII sites of pSEP vector by GenScript.

For cloning of DNA encoding DNA-PK catalytic subunit, since the gene size was too big to be synthesized in one piece, the gene was split into two pieces: DNAPK-NT (1-6504); and DNAPK-CT (6548-12387). Both gene sequences were codon optimized and unwanted restriction enzyme sites were removed. In addition, BamHI site was added at the 5′ end and additional DNA having the sequence of SEQ ID NO: 26 was added to the 3′ end of DNAPK-NT. Additional DNA sequences having the sequence of SEQ ID NO 27 and SEQ ID NO: 28 were added to the 5′-end and 3′-end of DNAPK-CT, respectively. These two custom-designed DNAs were synthesized by GenScript, and were sub-cloned into pUCD57 vector, yielding pUC57-DNAPK-NT and pUC57-DNAPK-CT. DNAPK-NT was then sub-cloned into BamHI and HindIII sites of pSEP vectors. DNAPK-NT and DNAPK-CT were combined to generate a full-length DNA-PK gene as follows: EcoRI and XbaI fragment from pSEP-DNAPK-NT, and PstI and HindIII fragment from pUC57-DNAPK-CT were combined using SLIC method, yielding, pSEP-DNA-PK vector.

Open reading frame (ORF) of MED12 from the yeast Saccharomyces cerevisiae (yMed12: GeneID 850442) was PCR amplified from the yeast genomic DNA using primer with complementary sequence of the BamHI region (yMed12_SEP_F; SEQ ID NO: 46) or HindIII region of the pSEP vector (yMed12_SEP_R; SEQ ID NO: 47), and cloned into pSEP vector by SLIC method (4).

Human BRCA1 gene (GeneID: 672) was PCR amplified using primer with complementary sequence of the BamHI region (BRCA1_SEP_F; SEQ ID NO: 48) or HindIII region of the pSEP vector (BRCA1_SEP_R; SEQ ID NO: 49), and cloned between BamHI and HindIII sites of pSEP vector by SLIC method (4).

CTCF gene from Drosophila melanogaster (GeneID: 38817) was PCR amplified from using primer with complementary sequence of the BamHI region (CTCF_SEP_F; SEQ ID NO: 50) or HindIII region of the pSEP vector (CTCF_SEP_R; SEQ ID NO: 51), and cloned between BamHI and HindIII sites of pSEP vector by SLIC method (4).

Human lymphoid-specific protein tyrosine phosphatase (Lyp) gene (PTPN22 GeneID: 26191) was PCR amplified from cDNA library using primer with complementary sequence of the BamHI region (Lyp_SEP_F; SEQ ID NO: 52) or HindIII region of the pSEP vector (Lyp_SEP_R; 53), and cloned between BamHI and HindIII sites of pSEP vector by SLIC method (4).

ORF of RRM3 gene from the yeast Saccharomyces cerevisiae (Rrm3p: GeneID 856426) was PCR amplified from the yeast genomic DNA, using primer with complementary sequence of the BamHI region (Rrm3_SEP_F; SEQ ID NO: 54) or HindIII region of the pSEP vector (Rrm3_SEP_R; SEQ ID NO: 55), and cloned into pSEP vector by SLIC method (4).

### Example 7. Virus Production and Protein Expression

Viruses were produced following the protocol of Fitzgerald et al. (2006)(5), and the viruses were stored by TIPS method described by Wasilko et al (2009)(6). The virus titers were measured, and the best eMOI for protein expression condition was determined by TEQC method (Imasaki et al., under revision). Proteins were expressed in 250 ml or 3 L Erlenmeyer cell culture flasks (Coming®), with 1.0×10⁶ cells/ml of Hi5 cells in 1 L ESF921 media (Expression systems) in optimized protein expression conditions (generally, eMOI between 0.5 to 4.0 with 96 hours incubation in 27 C.° on 100 rpm shaker). The cells were harvested by several rounds of centrifugation at 3000 rpm, frozen in liquid nitrogen, and stored at −80 C°.

### Example 8. Small-Scale Protein Purification

Frozen cell pellets were thawed on ice and were lysed by mixing with Lysis buffer (400 mM KCl, 50 mM Hepes (pH7.6), 10% Glycerol, 20 mM Imidazole) with 5 mM ß-mercaptethanol and 1× protease inhibitor mix (6 mM leupeptin, 20 mM pepstatin A, 20 mM benzamidine, and 10 mM PMSF). 10 ml of Lysis buffer in 50 ml culture was used for each cell pellet. After lysis, lysate was sonicated and centrifuged at high speed for 20 min (15,000 rpm in TOMY MX-301). The lysate was applied to 100 μl Ni resin (HIS-Select, Sigma-aldrich), and gently rotated at 4 C.° for 1 hour. The mixture was centrifuged at 8,000 g for 2 min, and the supernatant discarded. The resin was washed with 1 ml of high salt buffer containing 1 M KCl, 50 mM Hepes (pH7.6), 10% Glycerol, 5 mM ß-mercaptethanol, 20 mM Imidazole. The washing cycle was repeated 3 times, and after the last wash, the wash buffer was replaced with Lysis buffer with 5 mM ß-mercaptethanol. The proteins were eluted by 300 μl Lysis buffer with 300 mM Imidazole and 5 mM ß-mercaptethanol. The eluates were analyzed by SDS-PAGE.

### Example 9. Large-Scale Protein Purification for SEP Tagged Proteins

Frozen cell pellets were lysed using the same method as described above except that 200 ml lysis buffer in 1 L culture flasks was used. After lysis, cell lysate was sonicated and centrifuged by ultracentrifugation in a TL45 rotor (Beckman) at 35,000 rpm for 30 min. The lysate was applied to 2.5 ml of Amylose resin (NEB) and incubated for 30 min with gentle mixing. The mixture was passed through 15 ml size Econo-column (BioRad), and washed by adding 15 ml high salt buffer containing 1 M KCl, 50 mM Hepes (pH7.6), 10% Glycerol, and 5 mM DTT. The wash was repeated 3 times. Then, resin was washed with 15 ml of Lysis buffer with 5 mM DTT. After washing, the column was capped, and 2.5 ml of Lysis buffer with 5 mM DTT and 80 μg of 3C protease was added, mixed with pipet, and incubated at 4 C.° for overnight to elute target protein by digesting the SEP fusion protein. After digestion, column cap was opened, solution eluted, and 4 ml Lysis buffer with 5 mM DTT was added for washing every protein from the resin. The 6 ml elution was diluted by 18 ml of Buffer A (50 mM Hepes (pH7.6), 10% Glycerol, and 5 mM DTT), and applied to a 5 ml Hi-Trap Q HP (GE Healthcare Life Science) using BioRad FPLC. Proteins were purified by linear gradient elution followed by buffer A and Buffer B (50 mM Hepes (pH7.6), 1M KCl, 10% Glycerol, and 5 mM DTT). Eluted fractions were analyzed by SDS-PAGE. Target proteins were concentrated by Vivaspin20 (Sartorius) to less than 500 μl and applied to Superose6 10/300 GL (GE Healthcare Life Science) with Biorad FPLC. Elutions were analyzed by SDS-PAGE, and target proteins were harvested and concentrated by Vivaspin 20. The final target protein was harvested, and target protein concentration was analyzed by absorbance of OD280 by Nanodrop.

### Example 10. Solubility Assay

10×His-SUMO, 10×His-GST, 10×His-MBP, 10×His-AP, and 10×His-SEP tags were synthesized, and cloned into pUC57 vector by GenScript. For tagging NRPD1, tags were amplified by PCR using His_RBS_MC2_F (SEQ ID NO: 56) or His_MC2_F (SEQ ID NO: 92; dependent on whether an RBS site is present), and pre_NRPD1_R (SEQ ID NO: 58) primers, and the PCR products were gel purified. The purified PCR products encoding these tags were sub-cloned into BamHI and AscI sites of pSEP1-NRPD1 by SLIC method (4)—replacing SEP tag with tags above—yielding vectors harboring 10×His-SUMO, 10×His-GST, 10×His-MBP, 10×His-AP, and 10×His-SEP tagged NRPD1. The same cloning strategy was used to generate the vectors harboring 10×His-SUMO, 10×His-GST, 10×His-MBP, 10×His-AP, and 10×His-SEP tagged NRPD2. Expression viruses for tagged NRPD1 and NRPD2 were generated as described by Fitzgerald et al. (2006)(5). Proteins were expressed in 50 ml cell culture (see virus production and protein purification section). 1 ml cultures were harvested into 1.5 ml tubes, centrifuged, and stored at −80C°.

For western blotting, the cells were lysed w 100 μl of Lysis buffer containing 400 mM KCl, 50 mM Hepes (pH7.6), 10% Glycerol, 5 mM DTT, and 1× protease inhibitor mix (6 mM leupeptin, 20 mM pepstatin A, 20 mM benzamidine, and 10 mM PMSF) by pipetting. The lysate was centrifuged at high speed for 20 min (15,000 rpm in TOMY MX-301). The supernatant was mixed with NuPAGE sample Buffer (4×) (Thermofisher Scientific) and used for SDS-PAGE sample. The pellet from 100 μl lysate was resuspended with 2× of NuPGE sample buffer, and sonicated. Supernatant and pellet samples were subjected to Western Blot analysis, and probed with anti-NRPD1 or anti-NRPD2 antibodies (7). Detection was carried out using Dylight 680 goat anti-rabbit IgG (Thermo Scientific Pierce) and scanning with an Odyssey infrared imaging system (LI-COR Biosciences). Quantification was performed using ImageJ software (8).

### Example 11. Improvement of Vector Integration Efficiency

For all pSEPb vectors, the pUC1 origin of pFastBac1 (Invitrogen) was replaced with pMB1 origin from pRS322 (Addgene). However, the pSEPb vectors displayed low integration efficiency. To improve integration efficiency of pSEPb vectors (Table 1), vectors were remade using the original pFastBac1 origin of replication (pUC1), resulting in the pSEPa vectors (Table 1). As shown in FIG. 6, utilizing pFastBac1's original origin of replication resulted in a marked improvement in integration efficiency, as visualized by the increased number of white colonies (indicating integration) over blue colonies (no integration).

### Example 12. Construction of Exemplary pSEP Vectors

In some embodiments, an SEP tag comprises MBP and Solubility-Enhancing-Protein termed AP or SED tag followed by 3C protease site such that the entire SEP tag can be removed by 3C protease digestion. In some situations, the removal of the entire SEP tag from a newly synthesized protein can make the protein become insoluble. To avoid these situations, another version of a SEP tag was created by modifying the original tag as follows: 3C protease digestion site was placed in between MBP and AP (or SED) (see FIGS. 7 and 8). Referring now to FIG. 7, MBP will be removed by 3C protease digestion, and Solubility-Enhancing-Protein, AP or SED, is still intact, thereby providing solubility for a protein of interest. In part because AP or SED tag is disordered, AP or SED is unlikely to disrupt structure and function of proteins.

Referring now to FIGS. 7A and 8, pSEP20 (MBP-3C-AP), and pSEP21 (MBP-3C-SED) vectors have been generated. To make these vectors more versatile, pSEP22 and pSEP23 vectors were generated by adding TEV protease site and Twin-Strep tag in the C-terminus of a protein of interest.

Referring now to FIG. 9, a plant RPS5 protein was expressed and purified using the updated version of SEP system. RPS5 protein belongs to a class of intracellular receptors characterized by the presence of a Nucleotide Binding Domain and Leucine Rich Repeats (NLRs), which play a central role in the innate immune response by detecting pathogens inside both plant and human cells. RPS5 protein has proved to notoriously difficult to deal with because of its solubility issue. Although expression of RPS5 using the original version of SEP tag was successful, a removal of SEP tag made it insoluble. To solve the solubility problem of RPS5, the 2nd version of SEP tag were created. MBP-3C-AP-RPS5 fusion protein was successfully expressed in the insect cells. MBP tag was removed by 3C protease digestion, resulting in AP-RPS5 protein (FIG. 9A). AP-RPS5 fusion protein was examined by negative stain electron microscopy (EM) and a high abundance of particles of ˜10 nm in size having a uniform circular structure was identified (FIG. 9B)—the size and shape that were expected for a monomer of RPS5. In part because AP or SED tag is disordered, their appearance in EM has become invisible.

### Example 13. Solubility-Enhancing-Protein Assisted Protein Expression (SEP) System in E. coli (“eSEP System”)

The eSEP system enables to express large and often problematic proteins (molecular mass over 100 kDa) in E. coli. The key concept of SEP system lies in a development of solubility-enhancing-protein (SEP) tag, which facilitates expression, solubility and stability of a large target protein, thereby solving a long-standing problem in bioengineering. Referring now to FIGS. 10 and 11, pSEP vectors for protein expression in E. coli were generated—e.g., SEP5e and SEP6e. Both vectors comprise maltose-binding protein (MBP) and the synthetic solubility-enhancing protein termed “AP” or “SED” followed by 3C protease site (FIG. 10). Briefly, a gene encoding large and problematic protein can be cloned into the SEP vector having either SEP5e or SEP6e tag (FIG. 10). The SEP tag facilitates solubility and stability of proteins such that SEP tagged fusion protein can be recovered in soluble form. A target protein of interest can be purified by running it through an affinity-column followed by a 3C protease digestion (i.e., removal of SEP tag). The 3C protease digestion can be performed as an on-column digestion.

Referring now to FIG. 11A-11H, pSEP5e and pSEP6e vectors with a combination of two different origin of replications (pBR322, p15A), and three different antibiotics resistant genes (AmpR, ClmR, SpecR) from the scratch for future commercial use were generated. The expression of two largest subunits of plant RNA polymerase IV, NRPD1 and NRPD2, were examined using the eSEP system. Referring not to FIG. 12, SEP-tagged plant NRPD1 or NRPD2 was individually expressed in E. coli in SEP fusion protein forms: MBP-AP (or SED)-NRPD1 or MBP-AP (or SED)-NRPD2.

Unless indicated otherwise, all technical and scientific terms used herein have the same meaning as commonly understood by those of ordinary skill in biochemistry and genetic engineering.

All references to singular characteristics or limitations described herein shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

1. An expression vector encoding a solubility-enhancing polypeptide of about 75 to about 300 amino acids selected from glutamic acid (E), aspartic acid (D), and serine (S), wherein the solubility-enhancing polypeptide forms a disordered random coil, does not form any secondary structure, and E, D, and S are present in any ratio thereof.

2. The expression vector of claim 1, wherein the solubility-enhancing polypeptide comprises about 6 to about 27 acid patch subunits chosen from EEEDDDSSS (SEQ ID NO: 60), DDDEEESSS (SEQ ID NO: 61), and SSSEEEDDD (SEQ ID NO: 62).

3. The expression vector of claim 2, wherein each acid patch subunit is present in approximately equal numbers.

4. The expression vector of claim 2, wherein the acid patch subunits are linked via at least two glycine residues.

5. The expression vector of claim 2, wherein one or more residues from one or more acid patch subunits is modified to avoid formation of a secondary structure.

6. The expression vector of claim 4, wherein the solubility-enhancing polypeptide comprises no amino acid residues other than serine, glutamic acid, aspartic acid, and glycine.

7. The expression vector of claim 1, wherein the solubility-enhancing a polypeptide has at least 90% sequence identity to SEQ ID NO: 66.

8. The expression vector of claim 1, wherein the solubility-enhancing polypeptide comprises at least 50 three-amino acid repeats chosen from: serine-glutamic acid-aspartic acid; glutamic acid-aspartic acid-serine; and aspartic acid-serine-glutamic acid; and combinations thereof.

9. The expression vector of claim 1, wherein the solubility-enhancing a polypeptide has at least 90% sequence identity to SEQ ID NO: 4.

10. The expression vector of claim 1, wherein a polynucleotide encoding the solubility-enhancing polypeptide is operably linked to a promoter sequence.

11. The expression vector of claim 1, further comprising a multiple cloning site downstream of a polynucleotide encoding the solubility-enhancing polypeptide.

12. The expression vector of claim 1, wherein the expression vector further encodes a target protein, wherein the solubility-enhancing polypeptide and the target protein form a fusion protein.

13. The expression vector of claim 12, wherein the target protein has a size of about 100 kDa or greater.

14. The expression vector of claim 1, wherein the expression vector encodes a solubility-enhancing polypeptide linked to at least one protein tag.

15. The expression vector of claim 14, wherein the at least one protein tag is selected from the group consisting of: an affinity protein tag; a solubility-enhancing protein tag; and a yield-improving protein tag.

16. The expression vector of claim 15, wherein the at least one protein tag comprises a His tag and/or an MBP tag.

17. The expression vector of claim 16, wherein the His tag comprises about 6 to about 14 histidine residues.

18. The expression vector of claim 1, wherein the protein tags are separated by a linker peptide.

19. The expression vector of claim 1, wherein the solubility-enhancing polypeptide is linked to a protease recognition site.

20. The expression vector of claim 19, wherein the protease recognition site is an HRV 3C protease cleavage sequence.

21. The expression vector of claim 12, wherein the fusion protein includes a protease recognition site between the solubility-enhancing polypeptide and the target protein.

22. The expression vector of claim 11, further comprising an additional multiple cloning site.

23. The expression vector of claim 1, wherein the vector is a mammalian expression vector, a bacterial expression vector, or a baculovirus expression vector.

24. (canceled)

25. (canceled)

26. The expression vector of claim 1, wherein the expression vector comprises a polynucleotide having a nucleic acid sequence of any one of SEQ ID NO: 16, SEQ ID NO: 17, SEQ ID NO: 18, SEQ ID NO: 19, SEQ ID NO: 21, SEQ ID NO: 22; SEQ ID NO: 76; SEQ ID NO: 77; SEQ ID NO: 78; SEQ ID NO: 79; SEQ ID NO: 80; SEQ ID NO: 81; SEQ ID NO: 83; SEQ ID NO: 84; SEQ ID NO: 85; SEQ ID NO: 86; SEQ ID NO: 87; and SEQ ID NO: 88.

27. A method comprising:
a) providing the expression vector of claim 1, wherein the expression vector further encodes a target protein, wherein the solubility-enhancing polypeptide and the target protein form a fusion protein; and
b) expressing the fusion protein from the expression vector.

28. (canceled)

29. The method of claim 27, wherein the target protein has a size of about 100 kDa or greater.

30. The method of claim 29, wherein the target protein is selected from the group of proteins consisting of: BRCA1; LRRK2; DNA-PKcs; MED12; RRM3; mTOR; LYP; and CTCF.

31. The method of claim 27, wherein the fusion protein is expressed in a recombinant host cell.

32. The method of claim 27, further comprising isolating and purifying the fusion protein.

33. The method of claim 32, further comprising separating the target protein from the solubility-enhancing polypeptide.

34. The method of claim 33, wherein separation is achieved by protease cleavage at a protease recognition sequence.

35. A kit comprising an expression vector of claim 1, wherein the expression vector comprises a cloning site suitable for cloning a polynucleotide encoding a target protein.

36. The kit of claim 35, wherein the target protein has a size of about 100 kDa.

37. The kit of claim 35, wherein the expression vector further encodes an affinity protein tag, a yield-enhancing protein tag, or both an affinity protein tag and a yield-enhancing protein tag.

38. The kit of claim 37, wherein the kit further comprises an affinity chromatography column and buffers for purifying an affinity protein-tagged target protein.

39. The kit of claim 35, wherein the expression vector further encodes a protease recognition site.

40. The kit of claim 39, further comprising a protease corresponding to the protease recognition site.

41. The kit of claim 35, wherein the cloning site is a multiple cloning site.

42. The expression vector of claim 1, wherein E, D, and S are randomly or nearly randomly arranged in the solubility-enhancing polypeptide.

43. The expression vector of claim 1, wherein the solubility-enhancing polypeptide further includes one or more amino acids other than E, D, and S.

44. An expression vector encoding a solubility-enhancing polypeptide of about 150 to about 200 amino acids selected from glutamic acid (E), aspartic acid (D), and serine (S), wherein the solubility-enhancing polypeptide forms a disordered random coil, does not form any secondary structure, and E, D, and S are present in any ratio thereof.

45. The expression vector of claim 44, wherein E, D, and S are randomly or nearly randomly arranged in the solubility-enhancing polypeptide.

46. The expression vector of claim 44, wherein the solubility-enhancing polypeptide further includes one or more amino acids other than E, D, and S.
